(19) United States
(12) Patent Application Publication
(10) Pub. No.: US 2022/0339319 A1
(43) Pub. Date: Oct. 27, 2022

(54) NOVEL COMPOSITIONS AND METHODS FOR BONE GRAFTS AND FUSIONS

(71) Applicant: The Johns Hopkins University, Baltimore, MD (US)

(72) Inventors: Timothy Witham, Towson, MD (US); Ethan Cottrill, Baltimore, MD (US)

(21) Appl. No.: 17/763,909

(22) PCT Filed: Sep. 25, 2020
(86) PCT No.: PCT/US2020/052621

§ 371 Date: Mar. 25, 2022

Related U.S. Application Data
(60) Provisional application No. 62/906,295, filed on Sep. 26, 2019.

(51) Int. Cl. A61L 27/36; A61L 27/12; A61L 27/26; B29C 39/00; B29B 7/90

(52) U.S. Cl. CPC A61L 27/3633; A61L 27/12; A61L 27/26; A61L 27/365; A61L 27/3687; B29C 39/003; B29B 7/90; A61L 2430/38; A61L 2300/414; A61L 2430/02; B29K 2509/02

(57) ABSTRACT

The present invention pertains to novel bone graft substitute materials. These materials are porous, homogenously dispersed solid mixtures of calcium phosphate and pro-regenerative extracellular matrix (ECM)—and potentially any pharmaceutical agent and/or mineral—that have been infused with polydopamine. In some embodiments the bone graft materials have osteoinductive factors incorporated within them.

### REFERENCE TO RELATED APPLICATIONS

This application claims the benefit of U.S. Provisional Patent Application No. 62/906,295, filed on Sep. 26, 2019, which is hereby incorporated by reference for all purposes as if fully set forth herein.

### BACKGROUND OF THE INVENTION

Bone grafting is a surgical procedure used to create new bone. New bone is often needed in medicine and in dentistry. For example, when a fracture is not healing, or when insufficient bone is present for dental implants, health care providers may perform a bone grafting procedure to create bone to treat the patient. In addition, bone grafting is commonly used in spinal surgery—specifically, in a procedure called spinal fusion or spinal arthrodesis. In a spinal fusion procedure, bone grafting is used to create bone across a joint space to immobilize, or “fuse,” segments of the spine. This is done to treat neck pain, back pain, and spinal instability either with or without nerve compression. About 400,000 spinal fusion procedures are performed each year in the United States.

A bone graft is the material used in a bone grafting or arthrodesis/fusion procedure. Currently, the “gold standard” bone graft is autologous bone, which is bone that is taken from and used in the same individual being treated. In this way, bone is moved from one location to another in the body. Because this graft is bone, it readily forms bone in a new location. However, autologous bone is limited in supply—and using autologous bone often requires an additional surgery for obtaining the donor bone and may cause morbidity (e.g., pain/fracture) at the site of obtainment. For example, in spinal fusion surgery, autologous bone is often obtained from the bony pelvis or a rib for bone grafting purposes. This generally lengthens the time of surgery and causes additional pain to the patient.

Alternatives to autologous bone grafts comprise grafts that aim to reduce or replace the need for autologous bone in bone grafting procedures, while effectively and safely creating new bone. Autologous bone graft alternatives are either allograft bone or bone graft substitutes. Allograft bone is bone that is harvested from human cadavers, sterilely processed and transplanted into a recipient. Bone graft substitutes are all other bone graft materials, including derivatives of allograft bone (e.g., “demineralized bone matrix”) and laboratory-created materials. In general, allograft bone is less efficacious than autologous bone at creating new bone and is associated with a risk of immunogenic rejection and disease transmission. Bone graft substitutes, in contrast, offer expanded opportunities to create novel bone graft materials.

An example of a leading bone graft substitute is the Infuse™ Bone Graft, which is marketed by Medtronic. This bone graft substitute comprises two parts: (1) a Type I collagen sponge, derived from bovine Achilles tendon, and (2) a solution of bone morphogenetic protein-2 (BMP-2). To use, the collagen sponge is soaked in the solution of BMP-2 for a minimum of 15 minutes, and the wetted collagen sponge is then implanted for bone grafting. The Infuse™ Bone Graft typically results in similar rates of bony arthrodesis (e.g., spinal fusion) compared to autograft bone. However, this product is associated with relatively frequent adverse events from the BMP-2 dosage, including life-threatening complications, which led the United States Food and Drug Administration to issue a Public Health Notification about BMP-2 use in 2008. The potentially life-threatening complications associated with BMP-2 use in Infuse™ are believed to arise from the local bolus delivery of BMP-2, which may exceed one-million-times the normal physiological concentrations.

Another example of a bone graft substitute is Vitoss (Stryker), which is the #1 selling synthetic bone graft, with over 600,000 implantations worldwide. This bone graft is comprised solely of beta-tricalcium phosphate, which is similar to the primary mineral component of bone (i.e., hydroxyapatite). Vitoss is created with an interconnected porous structure resembling human cancellous bone. Because this bone graft substitute is comprised solely of calcium phosphate, it is brittle and, if used improperly, is susceptible to breakage during implantation. For spinal fusion, a more flexible bone graft that can be molded about the spine while holding together is desirable.

The extracellular matrix (ECM) is the physical microenvironment in which cells exist. It provides a substrate for cell anchorage, serves as a tissue scaffold, guides cell migration during embryonic development and wound repair, and plays key roles in tissue morphogenesis. In addition, the ECM is responsible for transmitting environmental signals to cells, which ultimately affects cell proliferation, differentiation, and death.

In general, the ECM is comprised of four major categories of biomolecules: (1) structural proteins (e.g., collagen and elastin), which in part provide strength and resilience; (2) glycosaminoglycans and proteoglycans (e.g., hyaluronate, chondroitin sulfate, and heparin sulfate), which in part cushion cells and sequester physiologically important proteins; (3) glycoproteins and other matricellular proteins (e.g., fibronectin, laminin, and osteopontin), which in part aid in cell adhesion, migration, growth, and differentiation; and (4) growth factors (e.g., fibroblast growth factors, transforming growth factor beta, and vascular endothelial growth factor), which regulate diverse cellular processes. Different combinations of these molecules tailor the matrix for different functions depending on the physiological needs and adaptations of the tissue. Further, ECMs may be isolated and used for clinical indications.

ECMs are commercially available for soft-tissue grafts, including ACell's MatriStem Urinary Bladder Matrix™ (UBM) for wound care and Cook Medical's Biodesign® Small Intestinal Submucosa (SIS) for a wide-range of general and reconstructive surgical indications. Both of these products are derived from porcine tissue (i.e., bladder or small intestines). Further, both are marketed to facilitate the restoration, or regeneration, of site-appropriate functional tissue because of the unique characteristics of the ECM, which allow for intimate cell contact and new tissue ingrowth. For example, because of their unique characteristics, ECMs may promote a pro-regenerative (e.g., “macrophage M2-phenotype”) remodeling healing response rather than a pro-inflammatory (e.g., “macrophage M1-phenotype”) healing response seen with other scaffolds. The MatriStem UBM products include an intact epithelial basement membrane on one surface and a lamina propria layer on the opposite surface; in contrast, the Biodesign® SIS product is comprised solely of the submucosal layer of proximal jejunum. Both products are processed from raw materials via mechanical and chemical means that minimize the loss of natural ECM components and structure, while yielding decellularized and sterile tissue. When implanted, these products are believed to be gradually remodeled in a way that helps the body restore itself.

MastriStem® products are available as sheets (e.g., MatriStem Wound Matrix, MatriStem Multilayer Wound Matrix, and MatriStem Burn Matrix) and particles (e.g., MatriStem MicroMatrix) with a particle size <500 micrometers. In contrast, the Biodesign® products are available as sheets (e.g., Biodesign Dural Graft, Biodesign Hernia Graft, and Biodesign Otologic Repair Graft), combination strip products (e.g., Biodesign Staple Line Reinforcement), plugs (e.g., Biodesign Fistula Plug Set), and cylinders (e.g., Biodesign Nipple Reconstructin Cylinder). Cook Medical separately offers a product called Powder Extracellular Matrix, which is micronized particles of porcine-derived SIS. CorMatrix® is another company that creates porcine-derived SIS sheets (e.g., CorMatrix ECM for Vascular Repair). All of these ECM products are initially created as sheets; the ECM is a layer, or sheet, isolated from the host tissue. Powdered versions may then be created from these sheets or sheet pieces (e.g., by cryogenically milling the sheets). Further, the indications for all of these products are for soft tissues; none are indicated for creating bone (e.g., as a bone graft). That is, there are no commercially available bone grafts involving pro-regenerative ECMs like UBM or SIS.

### SUMMARY OF THE INVENTION

The present invention pertains to the creation of a novel bone graft material that is a porous, homogenously dispersed solid mixture of calcium phosphate and ECM—and potentially any small molecule (e.g., drug) and/or mineral—that has been infused with polydopamine. A bone graft material of the present invention is created from a one-pot liquid ECM hydrogel solution at room temperature and neutral pH, which allows virtually any bioactive small molecule and/or mineral to be added to the bone graft substitute during the synthesis.

The materials of the present invention were designed and created to overcome the limitations of current leading bone graft substitutes. For example, primary limitations of the Infuse™ Bone Graft include its bio-disparate design and supraphysiologic burst release of growth factor (i.e., BMP-2). To solve this, the materials of the present invention involve a biomimetic scaffold, comprised of both inorganic (calcium phosphate) and organic extracellular matrix (ECM) components, similar to the composition of bone. This is in contrast to the Type I collagen sponge used in the Infuse™ sponge. Further, the addition of polydopamine confers a controlled and sustained growth factor release, which serves to overcome the bolus growth factor delivery method that complicates the existing Infuse™ product. In addition, the inventive biomimetic design confers flexibility to the graft, which serves to overcome the brittleness of the Vitoss product.

Further, these are the first biomimetic bone graft compositions that utilize ECM, which is a superior organic component source compared to Type I collagen alone. Finally, the synthetic strategy of a material of the present invention enables the creation of bone graft substitutes with a wide array of homogeneously dispersed known additives, including small molecules and minerals, since a material of the present invention is created from a one-pot liquid ECM hydrogel solution at room temperature and neutral pH.

For example, compounds such as antibiotics may be added to this solution during the synthetic process to yield a material of the present invention including an antibiotic. This bone graft substitute may decrease the risk of infection while effectively creating new bone or bone fusions. The capacity to create this large array of non-autologous bone graft substitutes is unique to the present invention and offers significant research and clinical value.

In some embodiments, the components of the materials of the present invention are naturally derived, including the expected metabolites during the resorption and replacement of the bone graft substitute with bone.

In accordance with a first embodiment, the present invention provides a biomimetic bone graft material comprising one or more calcium phosphate particles, extracellular matrix (ECM) particles comprising one or more of biomolecules including collagens, elastins, glycosaminoglycans, proteoglycans, glycoproteins, matricellular proteins, and a combination thereof in a mixture.

In accordance with a second embodiment, the present invention provides a biomimetic bone graft material comprising one or more calcium phosphate particles, extracellular matrix (ECM) particles comprising one or more of biomolecules including collagens, elastins, glycosaminoglycans, proteoglycans, glycoproteins, matricellular proteins, and a combination thereof, and further comprising one or more additional biologically active agents, in a mixture.

In accordance with a third embodiment, the present invention provides a method of making a bone graft material comprising the steps of: a) digesting ECM particles comprising structural proteins, glycosaminoglycans, proteoglycans, glycoproteins, matricellular proteins, growth factors, and a combination thereof, with a protease; b) neutralizing the digested ECM particles and forming a mixture comprising functional proteins or functional portions thereof; c) adding calcium phosphate particles to the mixture of c) and forming a homogeneously dispersed mixture; d) pouring the homogeneously dispersed mixture into a mold; e) incubating the mixture until it solidifies; f) lyophilizing the mixture forming a material; g) contacting the material with a solution of dopamine forming an activated material infused with polydopamine; and h) lyophilizing the activated material forming a bone graft material comprising an extracellular matrix.

In accordance with a fourth embodiment, the present invention provides the method above further comprising the step of contacting the bone graft material comprising an extracellular matrix with a solution containing a growth factor.

In accordance with a fifth embodiment, the present invention provides the method above further comprising the step of lyophilizing the bone graft material.

In accordance with a sixth embodiment, the present invention provides the method above further comprising the step of adding ECM particles before pouring the homogenously dispersed mixture into a mold.

### BRIEF DESCRIPTION OF THE DRAWINGS

FIG. 1 illustrates one method of making porcine-derived small intestinal submucosa (SIS) from raw material by physical separation of the tissue layers.

FIG. 2 illustrates one method of making porcine-derived urinary bladder matrix (UBM) from raw material by physical separation of the tissue layers.

FIG. 3 illustrates chemical processing of ECM pieces to decellularize and sterilize the material.

FIG. 4 illustrates lyophilized ECM (left: SIS; right: UBM).

FIG. 5 illustrates an embodiment of making ECM particles of specific size (as shown, <425 micrometers) by sieving cryogenically milled pieces of ECM.

FIG. 6 illustrates an embodiment for digesting ECM after 24 h in a dilute acid solution containing a protease (here, pepsin) while stirring.

FIG. 7 illustrates the addition of calcium phosphate particles following pH neutralization of the digested ECM solution, while stirring. Following pH neutralization, the mixture begins to gel/harden.

FIG. 8 illustrates an embodiment comprising a homogeneous mixture of ECM and calcium phosphate particles after 2 minutes of stirring following the addition of the calcium phosphate particles.

FIG. 9 illustrates pouring of homogenous mixture of ECM and calcium phosphate particles into a mold.

FIG. 10 illustrates an embodiment of a homogenous, gelled mixture of ECM and calcium phosphate particles after 1 hour of incubation at 37° C.

FIG. 11 illustrates lyophilized, porous, solid materials (i.e., bone grafts) comprising homogenously dispersed ECM and calcium phosphate particles and containing different ratios of ECM:calcium phosphate.

FIG. 12 illustrates the submersion of an embodiment of the present invention comprising a lyophilized, porous, solid material (i.e., bone graft) comprising homogenously dispersed ECM and calcium phosphate particles in a dopamine solution. The material, with a density greater than that of the dopamine solution, naturally sinks in the solution.

FIG. 13 illustrates bone graft embodiments of the present invention having different formulations. In some embodiments, the bone grafts of the present invention are a porous, homogenously dispersed solid mixture of calcium phosphate and ECM infused with polydopamine.

FIG. 14 illustrates some bone graft embodiments of the present invention being cut into pieces in preparation for bone grafting in a rat model of spinal fusion.

FIG. 15 illustrates exposure in a posterolateral, inter-transverse process rat model of spinal fusion, which is a clinically translatable model of spinal fusion. Here, the transverse processes of the lumbar vertebrae L4 and L5 and parts of the vertebral laminae can be seen.

FIG. 16 illustrates implantation of an embodiment of a bone graft of the present invention (shown in arrows). Note the flexibility and integrity of the bone graft, which can be molded about the spine.

FIG. 17 illustrates micro-computed tomography (micro-CT) scans obtained 8 weeks following posterolateral, inter-transverse process spinal fusion (rat model) at the lumbar vertebrae L4 and L5 using two different embodiments of the bone graft material of the present invention. In (17A), the bone graft material was created using SIS as the ECM source; in (17B), the bone graft material was created using UBM as the ECM source. Continuous, bridging new bone formation across the transverse process of L4 and L5 can be seen in (17A), whereas minimal new bone formation is seen in (17B).

FIG. 18 depicts the three embodiments of low oxysterol concentrations, high oxysterol concentration, and the biomimetic bone graft material without oxysterols.

FIG. 19 depicts pH paper indicators for the three embodiments of low oxysterol concentrations, high oxysterol concentration, and the biomimetic bone graft material without oxysterols.

FIG. 20 shows the chemical structures of various oxysterols useful in the bone graft materials of the present invention.

FIG. 21 is a photograph depicting the polydopamine infused biomimetic bone graft material of the present invention (dark pieces) compared to the Infuse™ Bone Graft collagen sponges (white pieces).

FIG. 22 is a photograph depicting the polydopamine infused biomimetic bone graft material of the present invention implanted in the spine of a rat.

FIG. 23 shows representative micro-CT images of each group (0.2, 2.0, and 20 micrograms of rhBMP-2 applied to either the polydopamine-infused biomimetic bone graft material of the present invention or collagen sponge) at 8 weeks postoperatively.

### DETAILED DESCRIPTION OF THE INVENTION

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “bone graft” is meant a material used in a bone grafting procedure.

By “bone grafting” is meant a surgical procedure used to create new bone or augment new bone formation.

By “biomimetic” is meant relating to or denoting synthetic methods or products that mimic biological or biochemical processes.

By “calcium phosphate particle” is meant a particle less than 500 μm, less than 300 μm, less than 200 μm, less than 150 μm, less than 100 μm, or less than 50 μm. The particle may have a size in the range of 20 μm to 50 μm, 30 μm to 100 μm, 50 μm to 150 μm, 100-200 μm, 150-300 μm, 200-400 μm, or 350-500 μm or a combination thereof. A range of particle sizes may be used in the present invention. For example, a bone graft of the present invention may use ⅓ calcium particles having a size of 20-50 μm, ⅓ calcium particles having a size of 150-300 μm, and ⅓ calcium particles having a size of 350-500 μm. A calcium phosphate may be further defined as any chemical entity comprising in part or in whole the following elements: calcium, phosphorous, and/or oxygen. For example, this includes monocalcium phosphate, dicalcium phosphate, tricalcium phosphate (e.g., beta-tricalcium phosphate), tetracalcium phosphate, octacalcium phosphate, amorphous calcium phosphate, dicalcium diphosphate, calcium triphosphate, apatite, and calcium pyrophosphate. In addition, a calcium phosphate may be in hydrated or anhydrous form (e.g., monobasic calcium phosphate monohydrate and dicalcium phosphate anhydrous). In addition, a calcium phosphate may be modified by various coatings (e.g., lipid-coated, plastic-coated, and/or polydopamine-coated) or be used as a coating for other chemical entities (e.g., calcium phosphate-coated metals, such as magnesium and/or titanium). In addition, a calcium phosphate as defined herein includes substituted calcium phosphates (e.g., silicate-substituted calcium phosphates, in which a proportion of the phosphate groups are substituted with silicate). In addition, a calcium phosphate as defined herein may be a chemical entity containing elements in addition to calcium, phosphorous, and/or oxygen, which includes bioactive glasses and glass-ceramics (e.g., chemical entities comprised of SiO₂—CaO—P₂O₅—Na₂O, like S53P4 and 45S5 bioactive glasses; and apatite-wollastonite containing glass ceramic).

By “disease” is meant any condition, disorder that damages, or interferes with the normal function of a cell, tissue, or organ. Examples of diseases treated by the methods and compositions of the present invention include fracture, neoplasm, instability (e.g., spinal instability), nerve root and plexus disorder, myelopathy, radiculopathy, osteoarthritis, spondylosis, spondylolysis, spondylolisthesis, atrophy, osseointegration failure, dental implant failure, degenerative disc disease, chronic pain, pseudarthrosis, major osseous defect, osteopenia, and osteoporosis.

By “extracellular matrix” or “ECM” is meant the physical environment in which cells exist. The ECM, in general, is comprised of four major categories of molecules: (1) structural proteins (e.g., collagen and elastin), which in part provide strength and resilience; (2) glycosaminoglycans and proteoglycans (e.g., hyaluronate, chondroitin sulfate, and heparin sulfate), which in part cushion cells and sequester physiologically important proteins; (3) glycoproteins and other matricellular proteins (e.g., fibronectin, laminin, and osteopontin), which in part aid in cell adhesion, migration, growth, and differentiation; and (4) growth factors (e.g., fibroblast growth factors and transforming growth factor betas), which regulate diverse cellular processes. Unique to ECM (as opposed to, e.g., collagen alone) is the ability to promote a pro-regenerative (e.g., “macrophage M2-phenotype”) rather than a pro-inflammatory (e.g., “macrophage M1-phenotype”) healing response.

By “ECM particle” is meant a particle having a size less than 700 μm, less than 600 μm, less than 500 μm, less than 400 μm, less than 300 μm, or less than 200 μm. The particle size may be from 50 μm to 500 μm, from 100 μm to 450 μm, or 300 to 450 μm. ECM particles comprise one or more of the following proteins including a collagen, an elastin, a glycosaminoglycan, a proteoglycan, a glycoprotein, a matricellular protein, and a growth factor. ECM particles are derived from mammalian tissue such as small intestine and bladder, as examples, and contain pro-regenerative tissue.

By “effective amount” is meant the amount required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “obtaining” as in “obtaining an agent” and the like includes synthesizing, purchasing, or otherwise acquiring the agent.

By “mineral” is meant chemical entity containing one or more ionic compounds (e.g., calcium phosphates, Na₂O—CaO—SiO₂).

By “prevent,” “preventing,” “prevention,” “prophylactic treatment” and the like refer to reducing the probability of developing a disorder or condition in a subject, who does not have, but is at risk of or susceptible to developing, a disorder or condition.

By “pro-regenerative” is meant promoting a constructive remodeling (e.g., “macrophage M2-phenotype”) healing response to facilitate the restoration, regeneration, and/or formation of site-appropriate functional tissue. Examples of pro-regenerative agents include ECMs (e.g., SIS and UBM); small molecules (e.g., interleukins-4 and 10); and cells (e.g. mesenchymal stem/stromal cells), including cell-derived components (e.g., mesenchymal stem/stromal cell-derived extracellular vesicles).

By “porous” is meant the property of having internal interconnected spaces or holes to facilitate new tissue (e.g., bone) ingrowth. Global porosity may be less than 80%, less than 70%, less than 60%, less than 50%, less than 40%, or less than 30%. Average interconnected pore size may be less than 200 μm, less than 150 μm, less than 100 μm, less than 50 μm, or less than 20 μm.

The terms “polypeptide,” “peptide” and “protein” are used interchangeably herein to refer to a polymer of amino acid residues. The terms apply to amino acid polymers in which one or more amino acid residues is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

By “reduces” is meant a negative alteration of at least 10%, 25%, 50%, 75%, or 100%.

A “reference” refers to a standard or control conditions such as a sample (human cells) or a subject that is free, or substantially free, of an agent, condition, or disease.

As used herein, the term “oxysterols” means a class of steroid derivatives, also called hydroxycholesterols, that have been studied for their potential osteoinductive effects. Common nomenclature of these molecules has yet to be standardized, with multiple “Oxy” names existing for some identical species. At their core, oxysterols are simply cholesterol oxidation products, which occur naturally in the human body. These compounds, such as 27-hydroxycholesterol (the most abundant circulating oxysterol in humans), have been described in a number of clinical contexts, though are perhaps best known for their role in cholesterol homeostasis, inflammation, and apoptosis.

Yet it was not until 2004 that the first description of the osteogenic activity of oxysterols was made, when it was demonstrated that specific oxysterols could induce osteogenic differentiation and matrix mineralization in in vitro preparations of murine mesenchymal stem cells.

TABLE 1
Various types of Oxysterols which can be used in the present invention

| Oxysterol name | Change from cholesterol |
|---|---|
| 20(S)-OHC | OH group at C₂₀(S) |
| 22(R)-OHC | OH group at C₂₂(R) |
| 22(S)-OHC | OH group at C₂₂(S) |
| Oxy4/Oxy34 | OH group at C20(S), single bond between |
| | C₅ and C₆, OH group at C₆(S) |
| Oxy18 | OH group at C₂₀(S), single bond between |
| | C₅ and C₆, OH group at C₆(S), deuterated |
| | carbons at C₂₂ and C₂₃ |
| Oxy21/Oxyl33 | OH group at C₂₀(S), single bond between |
| | C₅ and C₆, OH group at C₆(S), n-hexane at |
| | C₂₀(S) |
| Oxy49 | OH group at C₂₀(S), single bond between |
| | C₅ and C₆, OH group at C₆(S), double bond |
| | between C₂₅ and C₂₇ |

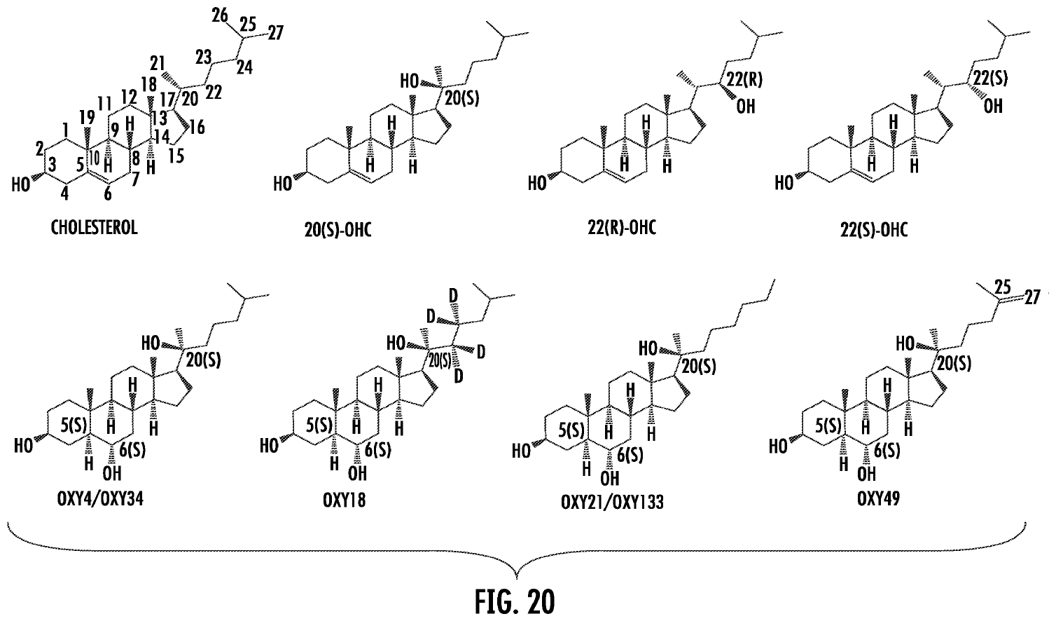

The structures of these compounds can be found in FIG. 20. It will be understood by those of ordinary skill in the art that other hydroxycholesterol derivatives and isomers other than these listed may be used in the compositions described herein.

Without being held to any particular theory, it is believed that oxysterol-mediated osteoinduction occurs via activation of the Hedgehog (Hh) signaling pathway, which is known to be essential for normal bone development. In conical Hh signaling, the transmembrane protein Patched (Ptc) inhibits Smoothened (Smo) activity. However, upon binding Hh ligand, Ptc is inactivated, allowing for Smo activation. Activated Smo increases levels of the active forms of Gli transcription factors, which translocate to the nucleus and upregulate multiple genes, including those key for bone formation. In a stereoisomer-specific manner, it is believed that oxysterols may directly activate Smo, thereby promoting Hh signaling and osteoinduction.

As used herein, the term “subject” is intended to refer to any individual or patient to which the method described herein is performed. Generally, the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

By “ranges” is meant to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “treat,” treating,” or “treatment,” and the like is meant reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

Such treatment (surgery) will be suitably administered to subjects, particularly humans, suffering from, having, susceptible to, or at risk of having a bone or cartilage defect, disease, fracture, disorder, or symptom thereof. Determination of those subjects “at risk” can be made by any objective or subjective determination by a diagnostic test or opinion of a subject or health care provider (e.g., genetic test, enzyme or protein marker, family history, and the like).

The materials of the present invention were designed and created to overcome the limitations of current leading bone graft substitutes. For example, primary limitations of the Infuse™ Bone Graft include its bio-disparate design and supraphysiologic burst release of growth factor (i.e., BMP-2). To solve this, the materials of the present invention involve a biomimetic scaffold, comprised of both inorganic (calcium phosphate) and organic extracellular matrix (ECM) components, similar to the composition of bone. This is in contrast to the Type I collagen sponge used in Infuse™. Further, polydopamine confers controlled and sustained growth factor release, which serves to overcome the bolus growth factor delivery method that complicates the Infuse™ product. In addition, the biomimetic design confers flexibility to the graft, which serves to overcome the brittleness of the Vitoss product. Further, this is the first biomimetic bone graft that utilizes ECM, which may be a superior organic component source compared to Type I collagen alone. Finally, the synthetic strategy of a material of the present invention enables the creation of bone graft substitutes with an infinite array of homogeneously dispersed additives, including small molecules and minerals, since a material of the present invention is created from a one-pot liquid ECM hydrogel solution at room temperature and neutral pH. For example, antibiotics may be added to this solution during the synthetic process to yield a material of the present invention including an antibiotic. This bone graft substitute may decrease the risk of infection while effectively creating new bone. The capacity to create this large array of bone graft substitutes is unique to the present invention and offers significant research and clinical value.

In some embodiments, the components of the materials of the present invention are naturally derived, including the expected metabolites during the resorption and replacement of the bone graft substitute with bone.

One embodiment of the present invention is a method of making extracellular matrix (ECM) particles. The method includes obtaining tissue from a mammal; decellularizing (and sterilizing) the tissue forming an ECM material; lyophilizing the ECM material forming a lyophilized ECM material; cryogenically milling the lyophilized ECM material; and forming ECM particles.

The decellularizing (and sterilizing) of the tissue may occur by various means including adding the tissue to an aqueous solution of ethanol and peracetic acid for a sufficient period of time with agitation. In one embodiment, the ECM can be made by shaking the 0.2 g of tissue per ml of solution comprising 95% distilled water, about 4% absolute ethanol and about 0.33% peracetic acid using an orbital shaker.

Suitable tissue used in the present invention includes small intestine tissue, bladder tissue, adipose tissue, stomach tissue, large intestine tissue, bone tissue, brain tissue, cartilage tissue, heart tissue, kidney tissue, liver tissue, pancreas tissue, trachea tissue, lung tissue, skeletal muscle tissue, pharynx tissue, esophagus tissue, spleen tissue, skin tissue, and/or tendon tissue. The tissue may come from most animals including pig. The tissue is used shortly after it is removed from the animal and is raw tissue, for example. The methods of the present invention may include additional steps such as sieving the ECM particles to a desired size.

Typically, the ECM particle composition comprises collagen, elastin, glycosaminoglycan, proteoglycan, glycoprotein, matricellular protein, various growth factors, and combinations thereof. In some embodiments, the ECM particle compositions have particles less than 700 μm in diameter and may be dry, for example.

The glycosaminoglycan in the ECM particle compositions ECM may be a hyaluronate, heparin, chondroitin sulfate, heparin sulfate, dermatan sulfate, keratan sulfate, and a combination thereof. The proteoglycan in the ECM particle compositions may be an aggrecan, perlecan, brevican, decorin, lumican, neurocan, versican, agrin, Type XVIII collagen, leprecan, proteoglycan 2, proteoglycan 3, hyaluronan and proteoglycan link protein, osteoadherin, prolargin, epiphycan, osteoglycin, chondroadherin, chondroadherin-like protein, nephrocan, podocan, podocan-like protein, testican, lubricin, endocan, and a combination thereof. The glycoprotein in the ECM particle compositions may be a fibronectin, laminin, biglycan, entactin, dermatopontin, colligin, nidogen, asporin, emilin, fibrillin, bone sialoprotein, matrilin, microfibrillar-associated protein, multimerin, nephronectin, osteonectin, SPARC-like protein, insulin-like growth-factor-binding protein, kielin/chordin-like protein, and a combination thereof. The matricellular protein in the ECM particle compositions may be a tenascin, thrombospondin, osteopontin, CCN family protein, fibulin, periostin, galectin, fibrinogen, vitronectin, ameloblastin, osteocalcin, cartilage intermediate-layer protein, dentin matrix acidic phosphoprotein, dentin sialophosphoprotein, matrix gla protein, latent transforming growth-factor beta-binding protein, or a combination thereof. The growth factors of the ECM particle composition may be a fibroblast growth factor, transforming growth factor, vascular endothelial growth factor, insulin-like growth factor, hepatocyte growth factor, epidermal growth factor, platelet derived growth factor, neurotrophin, erythropoietin, bone morphogenetic protein, interleukin, colony-stimulating factor, angiopoietin, or a combination thereof.

One embodiment of the present invention is a method of making a bone graft material comprising the ECM particle composition. The method comprises the steps of: a) digesting ECM particles comprising structural proteins, glycosaminoglycans, proteoglycans, glycoproteins, matricellular proteins, growth factors, and combinations with a protease; b) neutralizing the digested ECM particles forming a mixture comprising functional proteins; c) adding calcium phosphate to the mixture forming a homogeneously dispersed mixture; d) pouring the homogeneously dispersed mixture into a mold; e) incubating the mixture until it solidifies.

In some embodiments the method further comprises: f) contacting the material with a solution of dopamine forming an activated material infused with polydopamine and forming a bone graft material comprising an extracellular matrix.

In some embodiments the method further comprises: f) lyophilizing the mixture forming a material; g) contacting the material with a solution of dopamine forming an activated material infused with polydopamine; and h) lyophilizing the activated material forming a bone graft material comprising an extracellular matrix.

Other embodiments of the present invention comprise the step of contacting the bone graft material comprising an extracellular matrix with a solution containing a growth factor. The material at that point can be used as is, or it can then be lyophilized. Suitable growth factors used in the present invention include BMPs, calcitonin, parathyroid hormone, AB204, angiopoietin, erythropoietin, fibroblast growth factors, transforming growth factors, insulin, NEL-like protein 1, peptide B2A, insulin-like growth factor, vascular endothelial growth factor, platelet derived growth factor, hepatocyte growth factor, epidermal growth factor, interleukins, colony stimulating factors, neurotrophins, or combinations thereof. Additional growth factors used in the present invention include BMP-2, VEGF-165, PDGF-BB, and combinations thereof.

Other embodiments of the present invention include a step of adding a pharmaceutical agent or mineral before pouring the homogeneously dispersed mixture into a mold. Another embodiment of the present invention includes a step of adding ECM particles before pouring the homogenously dispersed mixture into a mold. Examples of proteases that may be used in the present invention include a pepsin, chymotrypsin, matrix metalloproteinase, collagenase, alcalase, papain, cathepsin, trypsin, and a combination thereof. In some embodiments, the pepsin used is derived from porcine gastric mucosa.

Any suitable shape or size of mold may be used. In an embodiment the mold used may have the dimensions of about 3 inches by about 2 inches though a variety of molds may be use that facilitate the attachment of a bone graft material to a bone, a broken bone, or a bony defect.

An example of a dilute base used in the present invention is sodium hydroxide and the solution has a concentration of about 5 to 25 mg ECM/mL, 5 to 15 mg ECM/mL, or 10 mg ECM/mL. Suitable incubating temperatures include from 20 to 50° C., 30 to 40° C., or 37° C. until the mixture solidifies. In some embodiments, the bone graft material is substantially free of water or dry. In some embodiments, the calcium phosphate particles have a size less than 200 μm, 150 μm, 100 μm, 90 μm, 80 μm, 70 μm, 60 μm, 50 μm, 40 μm, 30 μm, 20 μm, or 10 μm. The calcium phosphate particles may comprise a combination of 3-TCP and HA, for example. In some embodiments of the present invention, the bone graft material is in the range from 1 part ECM to 1 part calcium phosphate to 1 part ECM to 15 parts calcium phosphate. In some embodiments, the digestion of ECM particles occurs using 10 mg/ml of the ECM particle, 1 mg/mL of pepsin, and 0.01N HCl for between about 6 hours to about 72 hours. In an embodiment, the digestions occurs in 24 hours.

Another embodiment of the present invention is a bone graft material comprising one or more calcium phosphate particles; and ECM particles comprising one or more of collagens, elastins, glycosaminoglycans, proteoglycans, glycoproteins, matricellular proteins, growth factors and a combination thereof.

Another embodiment of the present invention is a bone graft material comprising calcium phosphate particles having a size less than 200 μm homogenously distributed in a matrix of ECM composition comprising one or more of the following: collagens, elastins, glycosaminoglycans, proteoglycans, glycoproteins, matricellular proteins, growth factors and combinations thereof.

In accordance with another embodiment, the present invention provides the bone graft material further comprising one or more of the following: pharmaceutical agents, minerals, polydopamine, growth factors, such as BMP-2, oxysterols, and a combinations thereof.

A pharmaceutical agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs” are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a biologically active agent may be used which are capable of being released from the subject composition, for example, into adjacent tissues or fluids upon administration to a subject.

Further examples of biologically active agents include, without limitation, enzymes, receptor antagonists or agonists, hormones, growth factors, autogenous bone marrow, antibiotics, antimicrobial agents, and antibodies. The term “biologically active agent” is also intended to encompass various cell types and transfected genes that can be incorporated into the compositions of the invention. Non-limiting examples of biologically active agents include following: adrenergic blocking agents, anabolic agents, androgenic steroids, anti-allergenic materials, anti-cholinergics and sympathomimetics, anti-coagulants, anti-convulsants, anti-infective agents, anti-inflammatory agents such as steroids, non-steroidal anti-inflammatory agents, anti-neoplastic agents, anti-pyretic and analgesic agents, anti-spasmodic agents, anti-thrombotic agents, biologicals, erythropoietic agents, estrogens, mineral supplements, mitotics, growth factors, nutritional substances, peripheral vasodilators, prostaglandins, vitamins, antigenic materials, and prodrugs.

In accordance with another embodiment, the present invention provides methods of bone grafting. These methods include the steps of implanting any one of the bone graft materials of the present invention in a subject having a medical condition requiring a bone graft at the site of the medical condition; and creating new bone growth in vivo. The methods of the present invention may be used to treat medical conditions including fractures; knee injuries; hip injuries; missing teeth replacement, tooth implants requiring bone for a dental implant; treatment of critical-sized bony defects; bone injuries or defects requiring a fusion procedure including, for example, foot, ankle, fingers, wrists, spinal fusion; or combinations thereof.

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Examples

Extracellular Matrix Particles

Extracellular Matrix (ECM) particles are used in the present invention. ECM particles may be commercially purchased (e.g., Powder Extracellular Matrix [Cook Biotech] and MicroMatrix® [ACell]), produced from commercially purchased non-particulate ECM products (e.g., MatriStem Wound Matrix, Biodesign Dural Graft, and CorMatrix® ECM for Vascular Repair), or produced using the methods of the invention. One embodiment of making ECM particles includes obtaining fresh porcine tissue (e.g., bladder and small intestines) from a slaughterhouse within 30 minutes of death of the animal. A “slaughterhouse inspector” from the United States Department of Agriculture oversees the obtainment of healthy, fresh tissue. Tissue from young market pigs (e.g., less than or equal to 300 lbs. in weight) of any breed may be used. The tissue is placed into a seal-tight plastic bag, cooled to approximately 32° C., and then transported to the laboratory (or similar facility) for processing.

At the laboratory, waste (e.g., urine and chyme) is removed from the tissue with running water. The tissue may be cut open and/or into smaller pieces to facilitate this process. For example, small intestines may be cut into approximately 12-inch-long pieces and each piece then cut open lengthways, such that the mucosa layer is visualized on one side and the serosa layer is visualized on the other side. Waste (chyme) may then be easily removed by pulling the tissue between a tight space created between one's fingers under running water. The tissue is rinsed with distilled water at room temperature, and then placed into a container with distilled water cooled to approximately 0° C. while the rest of the tissue(s) is similarly cleaned.

Of note, only proximal jejunum is used to create small intestine submucosa (SIS); the rest of the small intestine (e.g., duodenum and ileum) is not used. Practically, proximal jejunum is inferred to start 18 inches from the pyloric sphincter and include approximately 6 feet of tissue thereafter, or 1 foot proximal to the first appearance of Peyer's patches. The presence of Peyer's patches, or small circular patches of roughened areas in the tissue, indicates ileum tissue and should not be used in the making of SIS. Excess water is removed from the cleaned tissue by pulling the tissue between a tight space created between one's fingers. The tissue is then placed into a seal-tight plastic bag and frozen at −20° C. for between 12 and 96 hours.

The tissue is thawed in the sealed plastic bag under running tap water and then kept at approximately 0° C. The ECM layer is then obtained on a disinfected laboratory (or similar facility) bench by hand using: forceps, scissors, a scalpel, gauzes, and/or thin pieces of metal or plastic for scraping (e.g., polystyrene lids from 96 well-plates).

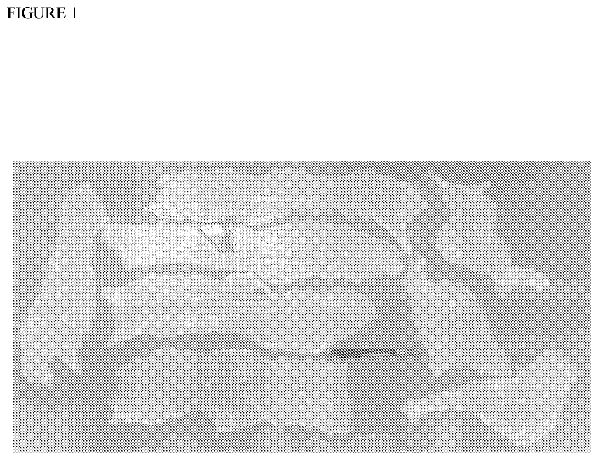

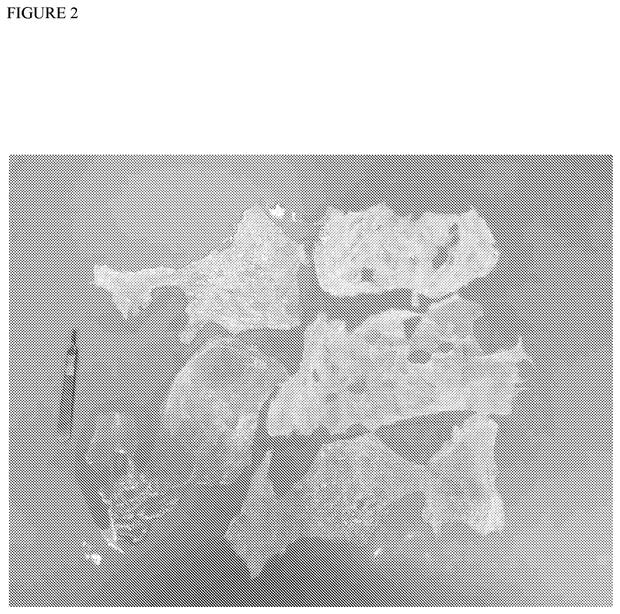

For example, for SIS, a 12-inch-long piece of proximal jejunum, cut open lengthways so as to expose the mucosa and serosa layers, is laid down flat (mucosa layer facing up) on the bench. The mucosa layer is then scraped away using the edges of two polystyrene lids from 96 well-plates pulling in opposite directions. The tissue is then flipped over (serosa side facing up) on the bench, and the serosa layer is similarly removed. Forceps, scissors, a scalpel and/or gauzes may be used to facilitate this process (e.g., by creating an initial small cut in the serosa layer, from which the serosa layer may be scraped away). Examples of the final ECM at this stage are shown in FIGS. 1 and 2 for SIS and UBM, respectively. The ECM is then cut into small pieces (approximately 1″×1″, Length×Width) using scissors to facilitate the chemical processing step (i.e., increase surface area and prevent clumping of ECM pieces, which facilitate decellularization and sterilization), which follows. The ECM pieces are temporally stored in a beaker containing distilled water at approximately 0 degrees Celsius until all ECM pieces from the available tissue have been generated.

The ECM is chemically processed to effectively decellularize and sterilize the material using a “decellularizing and sterilizing solution (DSS).” The DSS, used at an approximate concentration of 0.2 g ECM per mL DSS, is comprised of 95.67% distilled water, 4% absolute (200 proof) alcohol, and 0.33% peracetic acid (˜30% in dilute acetic acid). An approximate concentration is used because the mass of ECM is not precisely known because water is present in the ECM (i.e., excess water is removed from the ECM before weighing by pulling the pieces between a tight space created between one's fingers; however, the ECM is not fully dried at the time of weighing).

Table 1 provides guidance for making the DSS. This method may be scaled proportionately. An appropriately sized Erlenmeyer flask should be used for chemical processing based on the amount of ECM being processed and the total volume of DSS required. For example, for 60 grams of ECM, which corresponds to 1200 mL of DSS, a 2 L Erlenmeyer flask would be appropriate.

TABLE 1
Volumes of chemical reagents required for making the “decellularizing
and sterilizing solution (DSS)” based on the amount of ECM added to an
appropriately-sized Erlenmeyer flask for chemical processing.

| | Added Volume (mL) | | | |
|---|---|---|---|---|
| Added | | | Peracetic Acid | |
| ECM, | | Absolute | (~30% wt. % | |
| approximate | Distilled | Ethanol (200 | in dilute acetic | |
| grams | Water | proof) | acid) | Total |
| 20 | 383 | 16 | 1.33 | 400 |
| 40 | 765 | 32 | 2.67 | 800 |
| 60 | 1148 | 48 | 4.00 | 1200 |
| 80 | 1531 | 64 | 5.33 | 1600 |

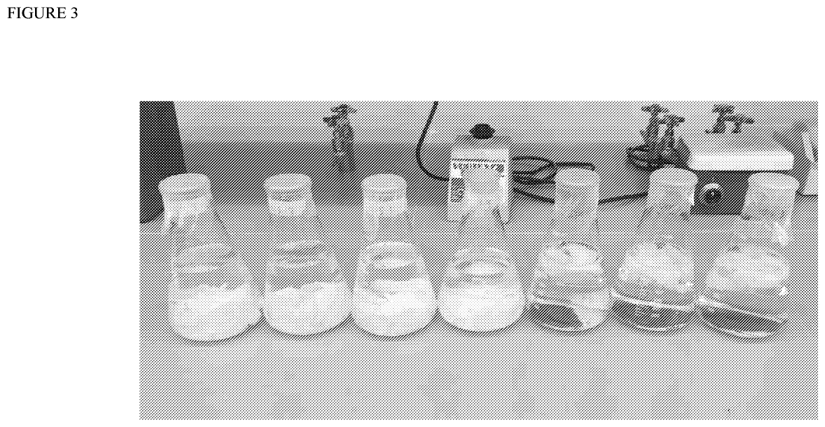

The weighted amount of ECM pieces are added to an appropriately-sized Erlenmeyer flask (chart above) and the required volume of DSS is then added to the flask. The flask is then sealed with Parafilm or similar means (FIG. 3). The flask containing the ECM and DSS is then shaken for 2 h at 200 RPM using an orbital shaker.

From here on, all work is performed in a way that maintains sterility of the ECM. The liquid is removed from the flask and replaced with the same volume of phosphate buffered saline (PBS). The flask is then resealed with Parafilm or similar and shaken on an orbital shaker for 15 min at 200 RPM. The liquid is then removed from the flask and replaced with the same volume of water. The flask is then resealed with Parafilm or similar and shaken on an orbital shaker for 15 min at 200 RPM. The liquid is then removed from the flask and replaced with the same volume of water. The flask is then resealed with Parafilm or similar and shaken on an orbital shaker for 15 min at 200 RPM. The liquid is then removed from the flask and replaced with the same volume of PBS. The flask is then resealed with Parafilm or similar and shaken on an orbital shaker for 15 min at 200 RPM. The liquid is then removed from the flask and replaced with the same volume of water. The flask is then resealed with Parafilm or similar and shaken on an orbital shaker for 15 min at 200 RPM.

The pH of the liquid is then confirmed to be neutral. If the pH is confirmed to be neutral, then the liquid is removed and the ECM pieces are transferred to one or more 50 mL conical tubes for lyophilization. The pieces are placed lengthways along the bottom ⅓ of the horizontally-held conical tube to maximize surface area for lyophilization. If the pH is not neutral (e.g., acidic), then the liquid is removed from the flask and replaced with the same volume of PBS. The flask is then resealed with Parafilm or similar and shaken on an orbital shaker for 15 min at 200 RPM. The liquid is then removed from the flask and replaced with the same volume of water. The flask is then resealed with Parafilm or similar and shaken on an orbital shaker for 15 min at 200 RPM. The pH of the liquid is then confirmed to be neutral, whereby, upon confirmation of neutral pH, the above-described steps regarding lyophilization follow. If the pH is still not neutral, the ECM pieces may be further washed with PBS followed by water, as described above, until neutral pH is obtained.

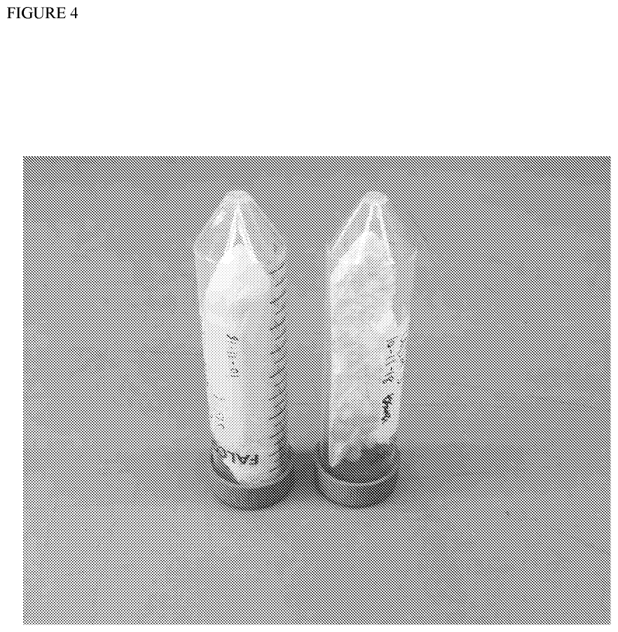

The tube containing the ECM is then laid horizontally in a −20° C. freezer for between 12 and 96 hours. The frozen ECM (−20° C.) is then lyophilized (vacuum approximately 0.1 mbar and the collector temperature is approximately −60° C.) for between 18 and 48 hours. Examples of final materials at this stage are illustrated for SIS and UBM in FIG. 4.

The lyophilized ECM is cut with scissors into small pieces (approximately 0.1 mm³) and then added to polycarbonate vials (e.g., Poly-Vial Set, SPEX SamplePrep) for cryogenic milling. The vials are filled approximately ⅓ full with ECM pieces. The ECM is then cryogenically milled in liquid nitrogen (e.g., SPEX SamplePrep, 6870 Freezer/Mill) using the following parameters: Cycles: 7, Pre-cool: 10 min, Run time: 2 min, Cool time: 3 min, Rate: 10 cycles/sec.

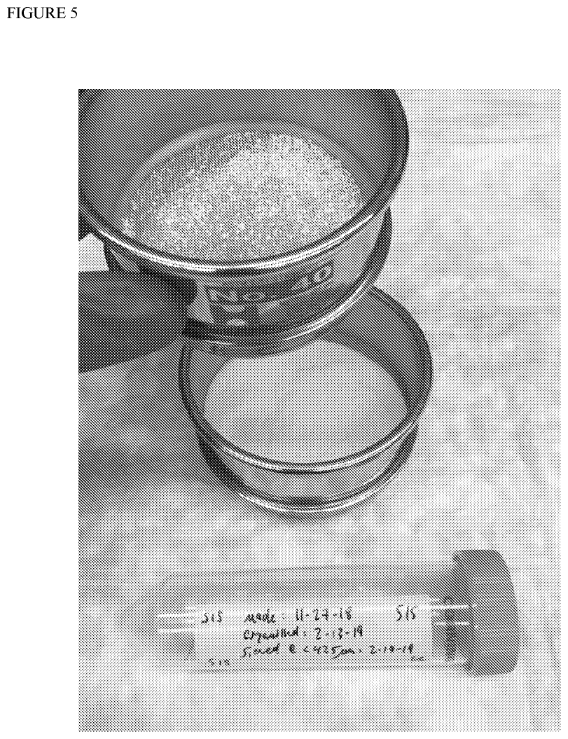

The cryogenically milled ECM is sieved through a mesh sieve (e.g., No. 40 mesh; 425-micrometers) (FIG. 5). The sieved ECM particles are then transferred to a 50-mL conical tube and stored at −80° C. until needed (for up to 1 year). The ECM particles may be characterized by a variety of means, including scanning electron microscopy (e.g. microarchitecture) and proteomics analysis. The composition of the ECM particles varies based on the source tissue.

As an example, UBM particles are comprised of more than eighty unique ECM molecules. This is in contrast to other bone graft substitute materials that comprise only one type of organic molecule (e.g., Type I collagen in the Infuse™ Bone Graft). The vast majority of the ECM molecules in UBM are collagens (approximately 97% by dry weight of UBM), including Types I, III, IV, V, VI, VII, VIII, IX, XI, XII, XIV, XV, XVIII, and XXI; the most abundant is Type I (approximately 65% of the total collagen content) followed by Type III (approximately 25% of the total collagen content). Additionally, proteoglycans and glycoproteins each constitute approximately 1% of the ECM molecules in UBM, whereas glycosaminoglycans, matricellular proteins, and growth factors together comprise the remaining approximately 1%. The most abundant of the proteoglycans, by dry weight of UBM, is decorin (approximately 0.3%) followed by lumican (approximately 0.2%). The most abundant of the glycoproteins, by dry weight of UBM, are dermatopontin and microfibrillar-associated protein 2 (each approximately 0.1%). Additionally, by dry weight of UBM, elastin constitutes approximately 0.05% of the ECM molecules in UBM. The most abundant matricellular proteins, by dry weight of UBM, are fibrinogen (approximately 0.1%) and fibulin (approximately 0.03%). Additionally, the most abundant growth factor, by dry weight of UBM, is transforming growth factor-beta (approximately 0.04%). It is believed herein that the biomimetic combination of biologically active molecules in ECMs will confer enhanced regenerative potential—and bone formation—to bone graft substitute materials, as described in the present invention, relative to the Type I collagen alone which is currently used in some commercially available bone graft substitute materials.

Bone Graft Material

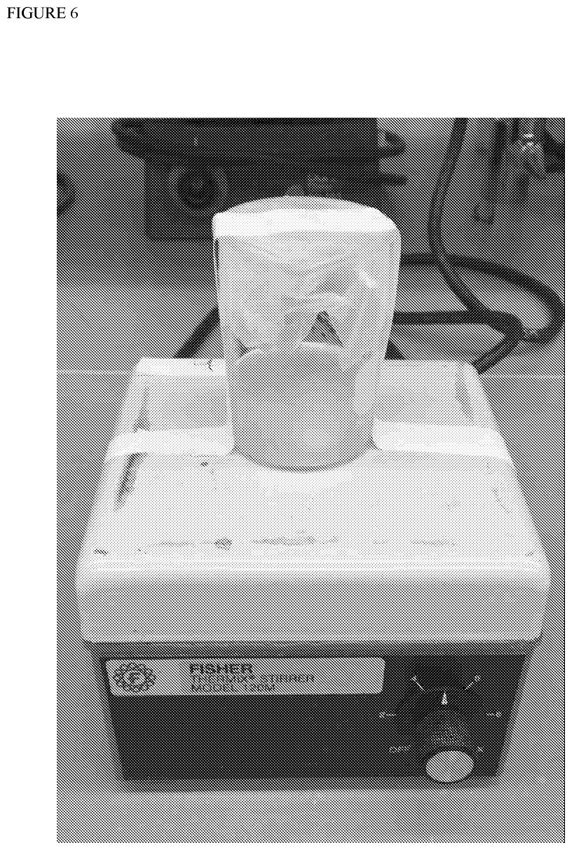

Bone graft materials of the present invention are prepared with ECM particle compositions described above. One embodiment of the present invention begins with digesting ECM particles with a protease in dilute acid at room temperature to create a digested ECM solution. For example, a protease (e.g., lyophilized pepsin from porcine gastric mucosa) is dissolved in a dilute acid (e.g., 0.1 N HCl) at a concentration of 1 mg protease/mL acid (e.g., 1 mg pepsin/mL 0.1 N HCl) in a sealed flask (e.g., beaker) while stirring at room temperature. The protease is fully dissolved when the solution is completely homogenous. Sieved ECM particles (created per the above) are added to this solution at a concentration of 10 mg ECM/mL solution. Alternatively, the protease solution may be added to sieved ECM particles in a flask (e.g., beaker). The flask is sealed with Parafilm or similar, and the mixture is allowed to stir (via magnetic stir bar and stir plate) at room temperature for about 24 h. The ECM should be completely dissolved, yielding a homogenous mixture without identifiable ECM particles, after 24 h (FIG. 6). As a variant, the mixture may be stirred for less than 24 hours (e.g., 1 h, 2 h, 3 h, etc.), which results in ECM particles with more native structure and function in the final bone graft material (paragraphs below).

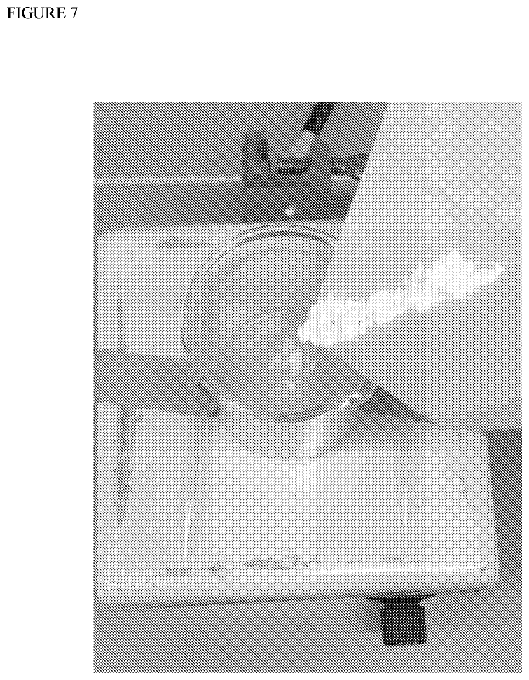

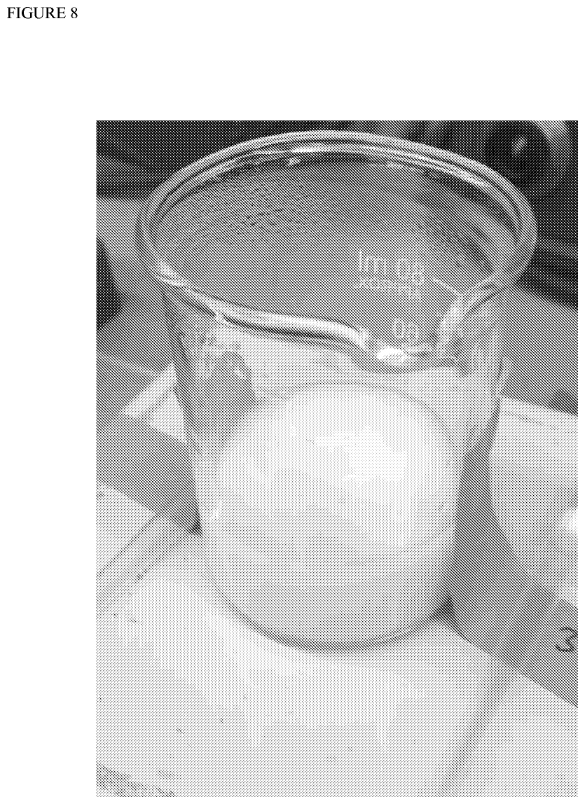

A dilute base (e.g., 0.1 N sodium hydroxide) is added dropwise to neutralize the digested ECM solution (approximately 1/10 the volume of acid described above) while stirring at room temperature. The pH is verified using pH strips, as the mixture at this point is too thick to use a liquid-based pH meter. Calcium phosphate particles, sieved through a mesh sieve (e.g., No. 325 mesh; 44-micrometers), are added to the mixture while stirring at a ratio from 1:1 to 1:20 (ECM:calcium phosphate) (FIG. 7). The mixture is allowed to stir for approximately 2 minutes to ensure homogenous mixing of the ECM and calcium phosphate particles; after 2 minutes, homogenous mixing is achieved (FIG. 8). As a possible variant, a buffer (e.g., 10×PBS) may be added to the mixture before neutralizing with dilute base to help ensure a neutral pH and/or dilute the mixture to aid in the subsequent homogenous mixing of the ECM and calcium phosphate particles. However, diluting the mixture too much (e.g., <7 mg ECM/mL solution) may cause the calcium phosphate particles to settle during the subsequent steps (paragraph below), resulting in poorly (i.e., not homogeneously) mixed ECM and calcium phosphate particles; this is to be avoided. Another possible variant is the addition of any powdered, granulized, liquid, and/or gas formulation of a pharmaceutical (e.g., drug) and/or mineral to the neutralized ECM solution in addition to or in place of the calcium phosphate particles, for similar homogeneous incorporation in the bone graft material by stirring. This ability to homogeneously incorporate any such additive into a bone graft material at room temperature and neutral pH is a unique characteristic of the present invention and offers significant research and clinical value. As examples, the pharmaceutical/mineral may include: antibiotics, growth factors, cytokines and chemokines, small molecules, carbohydrates, nucleic acids, proteins, lipids, cells and cell-derived products, metals and inorganic compounds, and metalorganic compounds, including naturally-derived and artificially-created substances.

In accordance with another embodiment, the present invention provides the use of separate, non-enzymatically digested and sieved ECM particles, including combinations of ECMs (e.g., SIS and UBM), which may also be added to the neutralized solution to homogenously incorporate ECM particles into the resulting bone graft material; the addition of these ECM particles may enhance the regenerative capacity of the resulting bone grafts as a result of providing native or near-native ECM protein structure and function. It is recognized that upon addition of any of the above-mentioned substances (e.g., pharmaceuticals, minerals, and/or ECM particles), neutral pH may need to be re-established, and the timing of gelation (paragraph below) may be affected. Finally, it is recognized that the properties of the final bone graft will change based on the composition of the material.

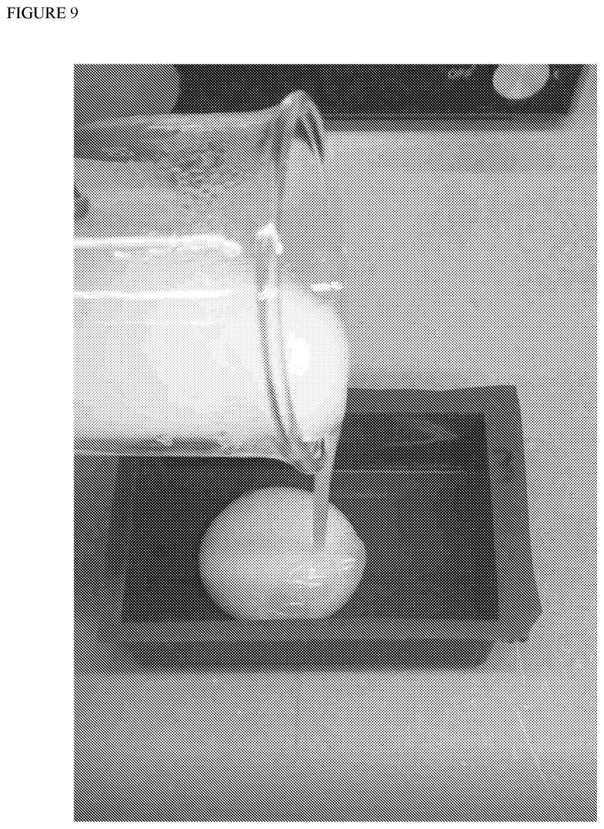

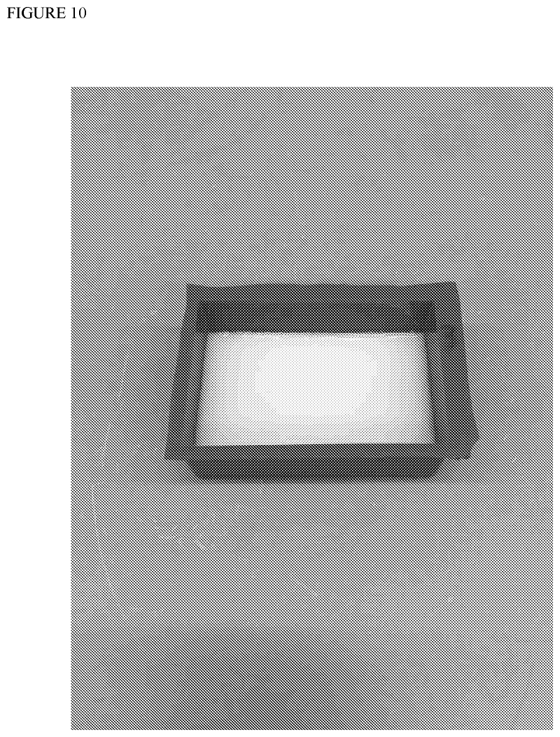

The homogenously dispersed mixture is poured into a mold, and the mold is tapped briefly to remove large air bubbles (FIG. 9). The mold containing the mixture is then placed into any type of sealed chamber (e.g., pipette tip box; at least 10-times the volume of the mixture). The chamber is then placed into an incubator at 37° C. for about an hour. After about an hour, the mixture is solidified to the consistency of gelatin or thicker (FIG. 10), depending on the amount of calcium phosphate and/or other additives added. As a possible variant, porogens may be added to the mixture before incubation to alter the pore structure of the bone graft material (paragraph below).

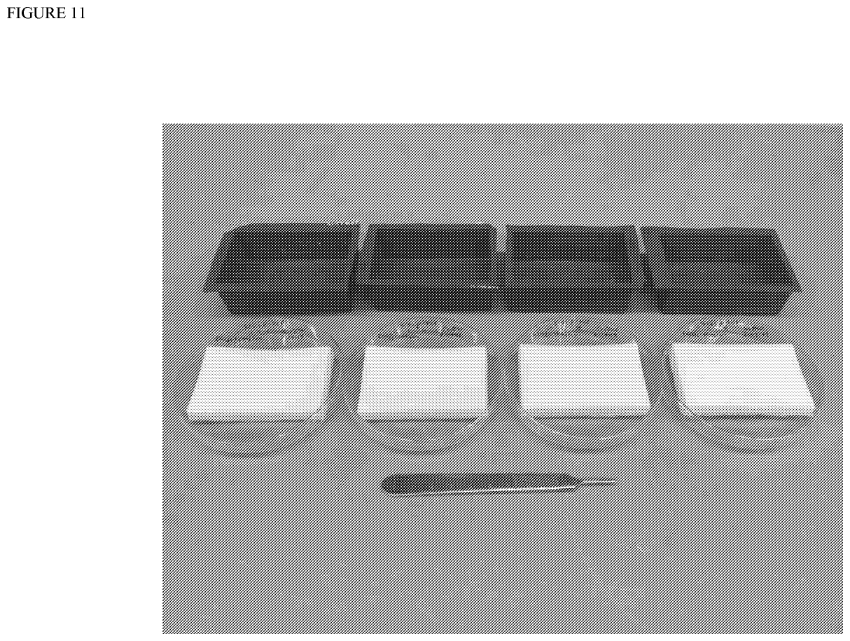

The mold containing the gelatin like mixture of ECM and calcium phosphate particles is transferred to a sealed container, which is then placed into a freezer at −20° C. for between 12 and 24 h. The frozen mixture still contained in the mold (−20° C.) is then lyophilized (vacuum approximately 0.1 mbar; collector temperature approximately −60° C.) for between 24 and 72 hours to dry the material and introduce pores. Examples of the final materials at this stage are shown in FIG. 11. As a possible variant, the freezing rate may be altered (e.g., near-instant freezing in liquid nitrogen) to alter the pore structure of the bone graft material.

It will be understood by those of ordinary skill in the art that the mold can have any shape and size for whatever grafting purpose is needed. The mold could have a long thin shape, or be round or oval or square or rectangular.

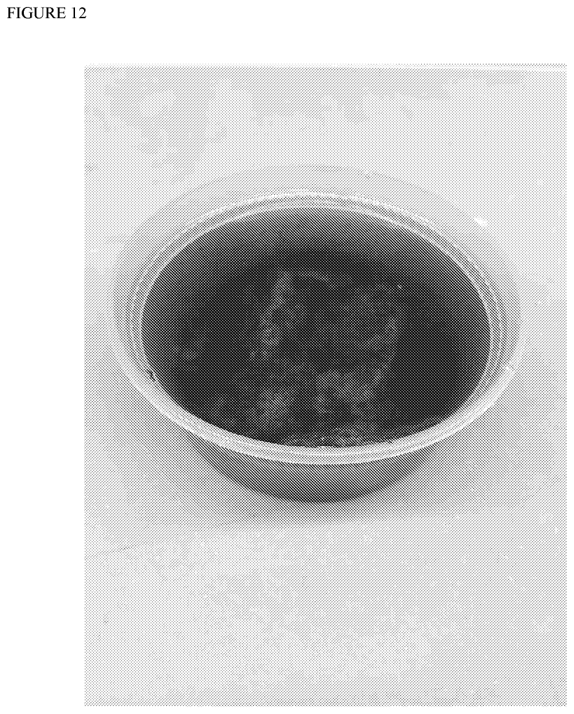

The lyophilized material created above is submerged in a dopamine solution (e.g., 2 mg dopamine HCl/mL of 10 mM tris buffer; adjusted to pH 8.5 with 0.1 N HCl or 0.1 N NaOH) at a concentration of 0.0075 g lyophilized material/mL dopamine solution for 3 h in a sealed container (e.g., polypropylene jar with lid) at room temperature (FIG. 12). To ensure uniform activation of the material with polydopamine, the container is flipped upside down once every 45 minutes. That is, the lyophilized material at a ratio of 1:2 (ECM:calcium phosphate) and greater (e.g., 1:3, 1:4, 1:5, etc.) is denser than the dopamine solution and sinks; flipping the container upside down periodically ensures uniform activation of the material with polydopamine.

The polydopamine-activated material is then repeatedly washed with distilled water to remove heterogeneous clumps of dopamine at room temperature. The supernatant containing heterogeneous clumps of dopamine is removed from the container and replaced with distilled water. The polydopamine-activated material is allowed to stand in the water for 10 minutes; then, the container is flipped upside down, and the polydopamine-activated material is allowed to stand in the water for 10 more minutes.

The supernatant containing heterogenous clumps of material is removed, and fresh distilled water is then added to the container. The polydopamine-activated material is allowed to stand in the water for 10 minutes; then, the container is flipped upside down, and the polydopamine-activated material is allowed to stand in the water for 10 more minutes. The above step is repeated (approximately 2 additional times) until no heterogeneous clumps of material are present and the water remains colorless after the end of the rinsing (i.e., after 20 minutes of rinsing).

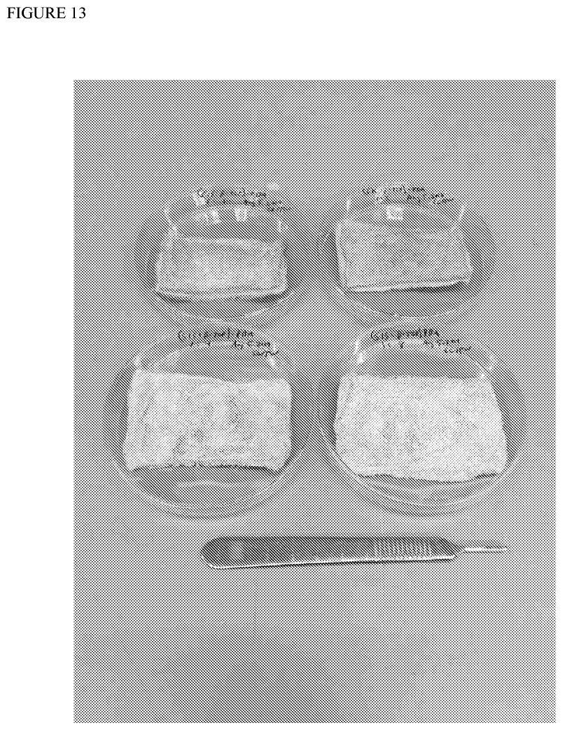

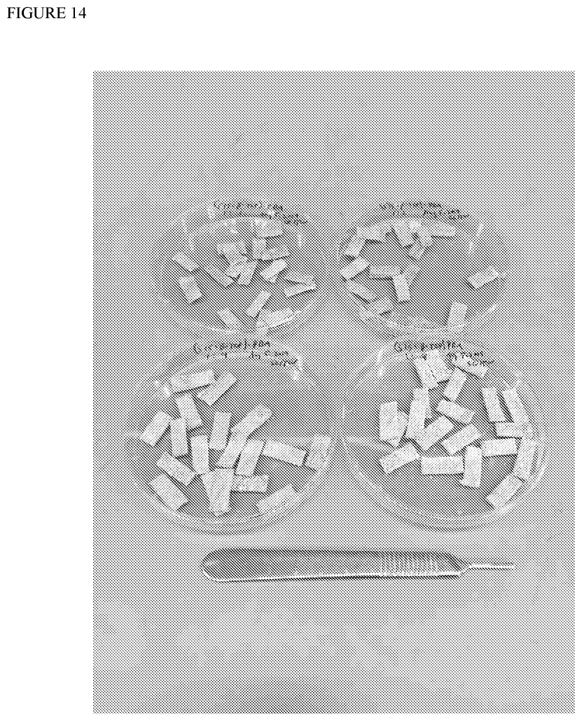

The polydopamine-activated material is transferred to a sealed container, which is then placed into a freezer at −20° C. for between 12 and 24 h. The frozen material (−20° C.) is then lyophilized (vacuum approximately 0.1 mbar; collector temperature approximately −60° C.) for between 24 and 72 hours to create the porous bone graft material. Examples of the bone graft materials at this stage are shown in FIGS. 13 and 14. As a possible variant, the freezing rate may be altered (e.g., near-instant freezing in liquid nitrogen) to alter the pore structure of a bone graft material.

Bone Graft Material with Growth Factors

In addition, as a possible variant, bone grafts of the present invention may be further activated with growth factors, which may have sustained and controlled release in the setting of polydopamine-activation. One or multiple different growth factors may be loaded onto the bone graft material as follows: the bone grafts are submerged in a solution (e.g., 10 mM tris buffer; pH 8.5) containing one or more growth factors (e.g., bone morphogenetic protein-2, vascular endothelial growth factor, and platelet-derived growth factor; concentration of 1 microgram growth factor/mL solution) in a sealed container at room temperature for 3 h. The container is periodically flipped over to ensure homogenous loading of the growth factors. Unconjugated growth factors are then rinsed away by replacing the growth factor solution with a solution that is free of growth factors (e.g., 10 mM tris buffer; pH 8.5) and allowing the growth factor-loaded material to stand in the growth factor-free solution for 10 minutes before flipping the container and allowing the material to stand for 10 additional minutes. The growth-factor loaded material is then transferred to a sealed container, which is then placed into a freezer at −20° C. for between 12 and 24 h. The frozen material (−20° C.) is then lyophilized (vacuum approximately 0.1 mbar; collector temperature approximately −60° C.) for between 24 and 72 hours to create a porous, growth factor-loaded version of a bone graft material. Similar to that described above, the freezing rate may be altered (e.g., near-instant freezing in liquid nitrogen) to alter the pore structure of the bone graft.

In accordance with yet another embodiment, the present invention involves mixing ECM particles (without enzymatic digestion) and calcium phosphate particles with or without pharmaceuticals and/or minerals (described previously) in a minimal amount of dilute glycerol (e.g., 10% in water), which is a non-toxic, odorless, colorless and biodegradable viscous liquid that is classified by the FDA as a “Generally Recognized as Safe” multiple purpose food substance. Further, glycerol is used as a carrier of particulate powders in some commercially available bone grafts, including Grafton® DBM and Optium® DBM. The mixture is transferred into a mold (e.g., 3″×2″), which is then placed into a sealed container. The container is then placed into a freezer at −20° C. for between 12 and 24 h. The frozen mixture is then lyophilized (vacuum approximately 0.1 mbar; collector temperature approximately −60° C.) for between 24 and 72 hours to dry the material and introduce pores. The lyophilized material is then activated with dopamine and re-lyophilized, as described above. All embodiments of the graft material may be coated with dopamine, and then re-lyophilized. In addition, all embodiments may include 3-D printing or electrospinning of the above-described bone graft components.

Scanning electron microscopy (e.g., microarchitecture) and biomechanical testing (e.g., elastic modulus) generally characterize bone graft materials of the present invention.

Methods of Bone Grafting Using the Materials—e.g., Spinal Fusion

A spinal fusion procedure is a surgical procedure used to create new bone about the spine to immobilize adjacent vertebrae. It is a surgical standard of care for patients with spinal instability, neoplasm, and/or spinal degenerative changes causing medication-refractory back and/or neck pain with or without spinal cord or nerve root compression. Successfully grafting bone about the spine is particularly challenging because, unlike in other applications of bone grafting (e.g., dental implants), the spine may be in motion during the grafting process. Small animal models of spinal fusion, including a posterolateral inter-transverse process fusion of the lumbar vertebrae L4 and L5, are clinically translatable models.

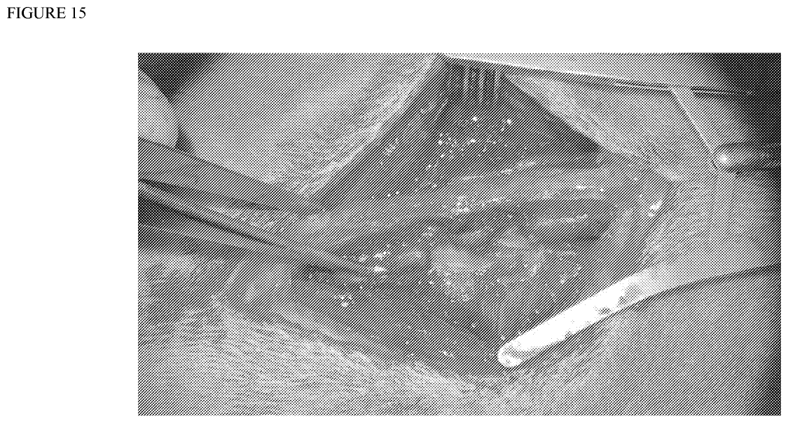

After Institutional Animal Care and Use Committee approval for the spinal fusion procedure has been received, a small animal (e.g., mouse, rat, or rabbit) is anesthetized using standard protocols. The hair on the back of the animal, in length being from approximately the thoracic vertebra T5 to the tail and in the width being that of the thoracic cavity, is then shaved using clippers. The skin is disinfected with 70% ethanol, and then sterilized with Betadine solution. A midline skin incision is then made beginning at approximately the thoracic vertebrae T9 and ending at the sacrum. Two paravertebral fascia incisions are then made, followed by careful dissection to expose the bony elements (e.g., transverse processes) of L4 and L5. Retractors and surgical dissecting instruments (e.g., forceps, scalpels, scissors, surgical curettes) may be used to facilitate the exposure (FIG. 15).

The bony elements are thoroughly cleaned of soft tissues which may interfere with bone grafting. In addition, the bony elements are decorticated (e.g., by cutting with a scalpel and/or using a drill) to expose red marrow; the red marrow may contain osteoprogenitor cells and osteoinductive factors (e.g., growth factors) to promote the spinal fusion. Further, decorticating the bony elements creates a vascular bed that may stimulate new vascular ingrowth in the implanted bone graft, facilitating the resorption and replacement of the bone graft with bone.

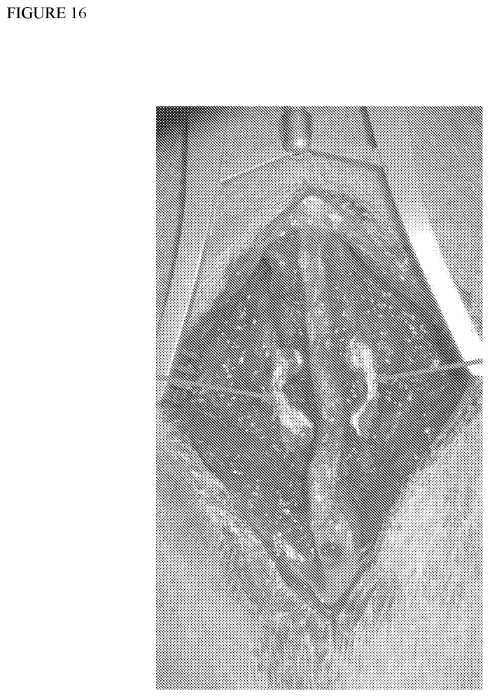

An embodiment of the bone graft material of the present invention is implanted over the decorticated bony elements and molded about the spine where new bone formation is desired. A bone graft which has enough flexibility to stay together while molding about the spine, without fragmenting, is desirable for spinal fusion (i.e., creating continuous, bridging bone between the adjacent vertebrae) (FIG. 16). The paraspinal muscles are then carefully returned to their normal, un-retracted position over the implanted bone graft, effectively sandwiching the bone graft between the decorticated bony elements and the paraspinal muscles. The fascia followed by skin is then closed using absorbable sutures, and the animal is then attended to for postoperative care while recovering from anesthesia (e.g., warming the animal and administering saline) before returning the animal to the cage.

The gold-standard method for evaluating spinal fusion clinically is via assessing for continuous, bridging bone formation between adjacent vertebrae via computed tomography—or, in animal models, micro-computed tomography. Continuous, bridging bone formation between the adjacent vertebrae is defined as a successful spinal fusion. A failed spinal fusion is defined as interrupted, or non-bridging, bone formation between the adjacent vertebrae. The time-point of evaluation ranges across animal models; in the described spinal fusion procedure in a rat model, commonly evaluated time points are 8 and 10 weeks.

Bone grafting may be performed similarly (including preparing the site for bone grafting, implanting the bone graft, and closing the surgical site and providing post-operative care) for a wide range of indications. These include the group consisting of a fracture; a knee problem requiring a knee replacement; a hip problem requiring a hip replacement; a missing tooth requiring bone for a dental implant; a bone issue requiring the treatment of critical-sized bony defects; a bone problem requiring a fusion procedure comprising a foot fusion, an ankle fusion, a finger fusion, a wrist fusion, and a spinal fusion; and a combination thereof.

A Method of Making SIS Particles

Fresh, healthy porcine small intestines were obtained from Wagner Meats, LLC, Mt. Airy, Md. within 30 minutes of slaughtering a young market Yorkshire hog (approximately 250 lbs. in weight). The small intestines were obtained with the assistance of a “slaughterhouse inspector” from the United States Department of Agriculture. The distal part of the stomach, including the pyloric sphincter, was also obtained for orientation purposes. The tissue was placed into a seal-tight, plastic bag and transferred to an insulated container filled with cold packs. The tissue was then transferred to the laboratory (Johns Hopkins Hospital, Baltimore, Md.).

The tissue was laid out on a disinfected laboratory bench. The pyloric sphincter was identified, and the first 18 inches of small intestines (distal to the pyloric sphincter; corresponding to the duodenum) was cut and removed from the rest of the small intestines. Next, a 12-inch-long piece of proximal jejunum was cut off, and the tissue then cut open lengthways. The chyme was removed from the tissue by pulling the tissue through a tight space created between my fingers under running water. The cleaned piece of small intestines was rinsed in distilled water and then temporarily stored in a beaker of distilled water cooled on ice. The next 12-inches of jejunum were cut off, cleaned and stored similarly. This process was repeated until the appearance of Peyer's patches, which signaled the end of the jejunum and beginning of the ileum. Any jejunum within 1-foot of the first appearance of Peyer's patches was identified and discarded. The rest of the jejunum was kept for the creation of SIS. Excess water from the cleaned and collected proximal jejunum was removed by pulling the pieces of tissue through a tight opening between my fingers. The pieces were then transferred to a seal-tight plastic bag and frozen for 36 hours at −20° C.

The proximal jejunum was then mechanically processed to obtain SIS. First, the laboratory bench was disinfected, and the following tools were prepared: scissors, forceps, scalpel, gauzes, and four polystyrene lids from 96-well plates. The frozen proximal jejunum was thawed in the sealed bag under running water, and, while still in the sealed bag, then placed on ice. A piece of proximal jejunum was obtained from the bag and laid mucosa-side up on the laboratory bench. The mucosa layer was scraped away by applying the edges of the polystyrene lids to the tissue in an opposite-direction pulling motion. Next, the tissue was flipped over (serosa-side up), and the muscularis and serosa layers were similarly removed using the polystyrene lids. The tissue was scraped with sufficient force to remove the mucosa and muscularis/serosa layers, but not too much that would tear the submucosa layer. A beautiful white, near-translucent material with cobweb-like connecting lines was obtained. To maximize subsequent decellularization and sterilization, this material was cut into small pieces (approximately 1″×1″, Length×Width), which were then transferred to a beaker containing distilled water, on ice. The process was then repeated for the remaining pieces of proximal jejunum.

The small pieces of SIS were then effectively decellularized and sterilized. First, excess water was removed from the pieces by pulling the pieces through a tight space between one's fingers, and then all the pieces were weighed. The obtained weight was approximately 60 g. Accordingly, 1200 mL of “decellularization and sterilizing solution (DSS)” was made, comprising 1148 mL (95.67%) of distilled water, 48 mL (4%) of 200-proof ethanol, and 4 mL (0.33%) of peracetic acid (from 32 wt. % in dilute acetic acid). The peracetic acid, which is a corrosive substance, was handled in a fume hood. A 2-L Erlenmeyer flask, previously cleaned and autoclaved, was obtained. The ECM (60 g) was then added to the Erlenmeyer flask, followed by the 1200 mL of the DSS. The flask containing the SIS and DSS was sealed with Parafilm, and then shaken for 2 h at 200 RPM and room temperature using an orbital shaker (VWR Symphony Model 5000I/R). Hereafter, effective sterility was maintained via tools and techniques.

The decellularized and sterilized SIS was then washed. First, the liquid was removed from the flask by pouring it off, and 1200 mL of PBS was then added to the flask. The flask was then re-sealed and then placed back onto the orbital shaker for 15 minutes at 200 RPM. The liquid was then removed, and 1200 mL of water was added to the flask. The flask was then re-sealed and then placed back onto the orbital shaker for 15 minutes at 200 RPM. The liquid was then removed, and 1200 mL of water was added to the flask. The flask was then re-sealed and then placed back onto the orbital shaker for 15 minutes at 200 RPM. The liquid was then removed, and 1200 mL of phosphate buffered saline was added to the flask. The flask was then re-sealed and then placed back onto the orbital shaker for 15 minutes at 200 RPM. The liquid was then removed, and 1200 mL of water was added to the flask. The flask was then re-sealed and then placed back onto the orbital shaker for 15 minutes at 200 RPM. The pH was confirmed at 6.9. The liquid was then removed, and the ECM pieces were transferred to two 50-mL conical tubes for lyophilization. The ECM pieces were placed lengthways along the bottom ⅓ of the horizontally-held conical tubes to maximize surface area for lyophilization.

The two conical tubes containing the SIS were then placed horizontally in a −20 degrees Celsius freezer for 18 hours. The frozen ECM (−20° C.) was then lyophilized (Labconco Freeze Dry System/Freezone 4.5; vacuum approximately 0.1 mbar; collector temperature approximately −60° C.) for 36 hours, yielding two soft white SIS materials in elongated semi-cylindrical shapes. The mass of SIS obtained was 2.5 g.

The lyophilized SIS was then cryogenically milled. First, the SIS was cut into small pieces of approximately 1 mm³ in a cell/tissue culture hood. The small pieces were then transferred into polycarbonate vials (Small Poly-Vial Set, SPEX SamplePrep), approximately ⅓ full and then cryogenically milled (SPEX SamplePrep, 6870 Freezer/Mill) using the following parameters:
i. Cycles: 7
ii. Pre-cool: 10 min
iii. Run time: 2 min
iv. Cool time: 3 min
v. Rate: 10 cycles/sec
   The process was repeated until the entire SIS was milled. The powder was collected in a 50-mL conical tube. The cryogenically milled SIS was then sieved through a No. 40 (425-micrometer) mesh sieve, transferred to a 50-mL conical tube, and then stored at −80° C. The mass of sieved SIS obtained was 2.2 g.

An Alternative Embodiment for Making the Bone Graft Material (SIS as the ECM)

Lyophilized pepsin from porcine gastric mucosa (22.5 mg; 3,200-4,500 units/mg protein) was dissolved in 22.5 mL of 0.1 N HCl (1 mg pepsin/mL 0.1 N HCl) in a 50-mL beaker equipped with a magnetic stir bar and sealed with Parafilm. The solution was stirred until the protease had completely dissolved and the solution was observed to be homogeneous (about 5 minutes). SIS (225 mg; 10 mg SIS/mL 0.1 N HCl), sieved to below 425 micrometers, was then added to the above solution. The beaker was then re-sealed with Parafilm and allowed to stir on a stir plate at room temperature for 24 h, at which point the ECM had completely dissolved and a near-colorless, homogenous viscous mixture without identifiable ECM particles was present. Dilute sodium hydroxide (2.22 mL; 0.1 N) was then added dropwise to neutralize the mixture while stirring. The solution was allowed to stir for 90 seconds at room temperature, after which the pH was confirm to be neutral. Next, 1800 mg of beta-tricalcium phosphate, sieved below 44 micrometers, (1:8 ratio of SIS:beta-tricalcium phosphate), was added to the neutralized mixture while stirring. The mixture was allowed to stir for 2 minutes, which resulted in a homogenous mixture of SIS and calcium phosphate particles, without distinction between the particles. It was observed that the mixture was beginning to gel/harden during this time.

The homogeneously dispersed mixture of SIS and beta-tricalcium phosphate was then poured into a 2″×3″ mold (Freshware SP-100RD). The mold was briefly tapped to remove air bubbles from the mixture, and then placed into a sealed Pipette tip box. This box was then placed into an incubator at 37° C. for 1 h, after which time the white mixture had solidified to the consistency of thick gelatin. The mold containing the gelatin-like homogenous mixture of SIS and beta-tricalcium phosphate was then transferred to a sterile bag. The bag was sealed and then placed into a −20° C. freezer for 16 h. The frozen mixture contained in the mold (−20° C.) was then lyophilized (Labconco Freeze Dry System/Freezone 4.5; vacuum approximately 0.1 mbar; collector temperature approximately −60° C.) for 36 hours, yielding a beautiful white, porous, foam-like material containing homogenously dispersed SIS and beta-tricalcium phosphate.

The lyophilized material created above was then activated with polydopamine. First, the material was submerged in 270 mL of a dopamine solution (2 mg dopamine HCl/mL of 10 mM tris buffer; pH 8.5; 0.0075 g lyophilized material/mL dopamine solution) in a plastic container with lid for 3 h at room temperature. To ensure uniform activation of the material with polydopamine, the container (and bone graft material) was flipped upside down once every 45 minutes. It was noted that the lyophilized material sank in the prepared dopamine solution. The polydopamine-activated material was then repeatedly washed with distilled water to remove heterogeneous clumps of dopamine at room temperature. Namely, the supernatant dopamine solution was removed and replaced with distilled water. The polydopamine-activated material was allowed to stand in the water for 10 minutes; the container was then flipped upside down, and the polydopamine-activated material was allowed to stand in the water for 10 additional minutes. The supernatant was then replaced with fresh distilled water, and the rinsing process repeated for three additional times, after which point no heterogeneous clumps of material were observed and the water remained colorless. The polydopamine-activated material was then transferred to a petri dish, which was then placed into a sterile plastic bag. These were placed into a −20° C. freezer for 16 h. The frozen material (−20° C.) was then lyophilized (Labconco Freeze Dry System/Freezone 4.5; vacuum approximately 0.1 mbar; collector temperature approximately −60 degrees Celsius) for 48 h, yielding a bone graft material.

Preliminary analyses support that 1) a 1:2 ratio of ECM to calcium phosphates promotes greatest bone growth (which corresponds to the most “biomimetic” composition, as native bone is comprised of ˜⅓ organic components and ˜⅔ inorganic components) and 2) polydopamine further promotes bone growth.

A Method of Bone Grafting (Spinal Fusion)

A posterolateral inter-transverse process spinal fusion of the lumbar vertebrae L4 and L5 in a rat model was performed after receiving Institutional Animal Care and Use Committee approval. A male Sprague Dawley rat at approximately 11 weeks of age was used. The rat was weighed to calculate the dosage of anesthetic (xylazine and ketamine, per standard protocol) required. The mass of the rat was 370 g, corresponding to 350-microliter of required anesthetic. The rat was injected with the anesthetic via intraperitoneal injection and then returned to its cage for the sedative to take effect. After approximately 5 minutes, the rat was observed to be fully asleep, as confirmed via lack of toe-pinch reflex. The hair on the back of the rat, in length being from approximately the thoracic vertebra T5 to the tail and in the width being that of the thoracic cavity, was then shaved using clippers. The skin was then disinfected with 70% ethanol, and then sterilized with Betadine solution.

A midline skin incision was made beginning at approximately the thoracic vertebrae T9 and ending at the sacrum using an 11-blade. Two paravertebral fascia incision were then made using the same blade, followed by careful dissection to expose the bony elements (e.g., transverse processes) of the lumbar vertebrae L4 and L5. Retractors and surgical dissecting instruments, including forceps, scissors, and surgical curettes, were used to facilitate the exposure of the bony elements of L4 and L5. The transverse processes and lamina of L4 and L5 were then decorticated by cutting the bony elements with a 12-blade until punctate bleeding was observed. Afterwards, two pieces of bone graft material (i.e., SIS:beta-tricalcium phosphate in a 1:8 ratio), previously cut from a larger piece of graft (size per piece: 17 mm×7 mm×3 mm, L×W×H), were implanted over the decorticated bony elements, one per side, and molded about the spine where new bone formation was desired. Excellent flexibility and integrity of the grafts were noted on molding the material, especially when wetted with bone marrow from the decortication. The paraspinal muscles were then carefully returned to their normal, un-retracted position over the implanted bone grafts, effectively sandwiching the bone grafts between the decorticated bony elements and the paraspinal muscles. The fascia followed by skin was then closed using absorbable 3-0 sutures (Vicryl). The rat was administered 1 mL of normal saline subcutaneously and placed onto a heating pad until fully awake, after which point the animal was transferred to a clean cage with readily available access to food and water.

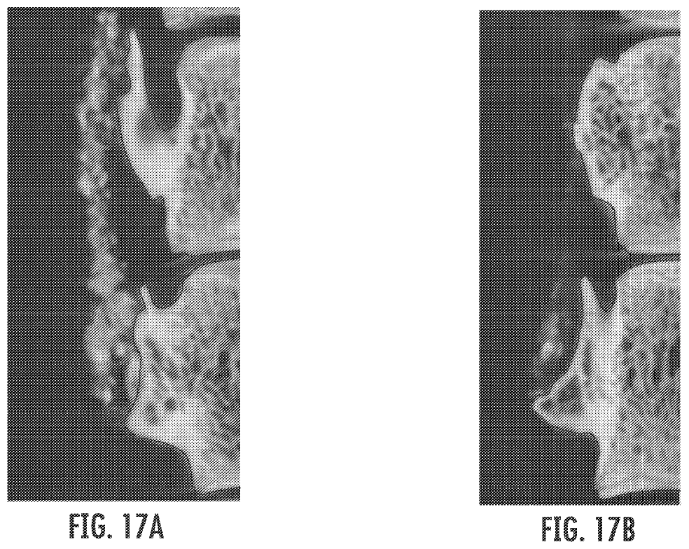

ECM can be isolated and used from any tissue/organ, and any such ECM is contemplated in the scope of the present invention. For example, our preliminary work suggests that SIS may be a superior source of ECM relative to UBM in the present invention. That is, versions of bone graft materials were created as described herein, with the only difference being the source of ECM—either from SIS or UBM. The SIS version demonstrated superior bone formation in a clinically translatable rat model of spinal fusion compared to the UBM version, as shown in FIG. 17.

Bone Graft Materials Comprising Oxysterols

In accordance with some embodiments, the bone graft materials incorporate various oxysterols.

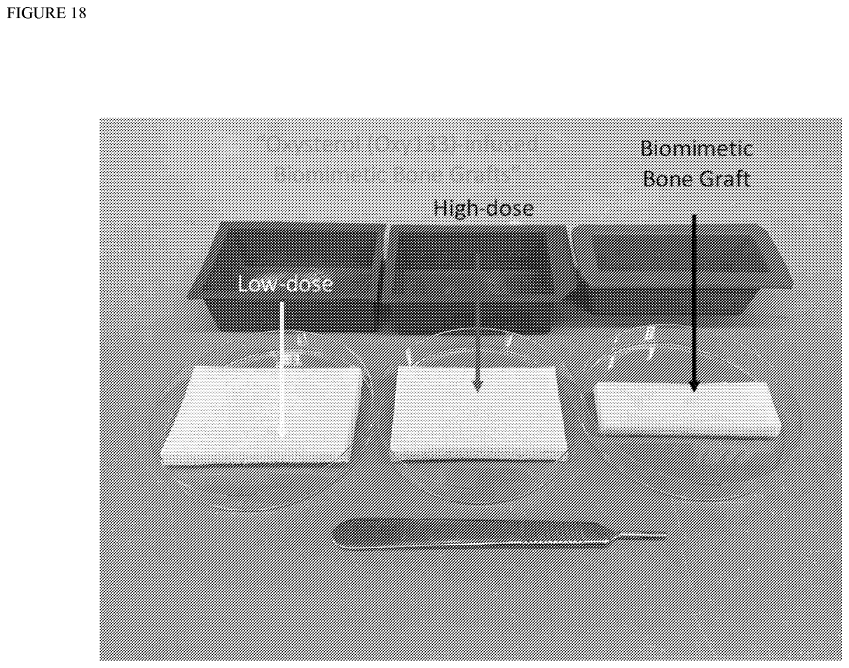

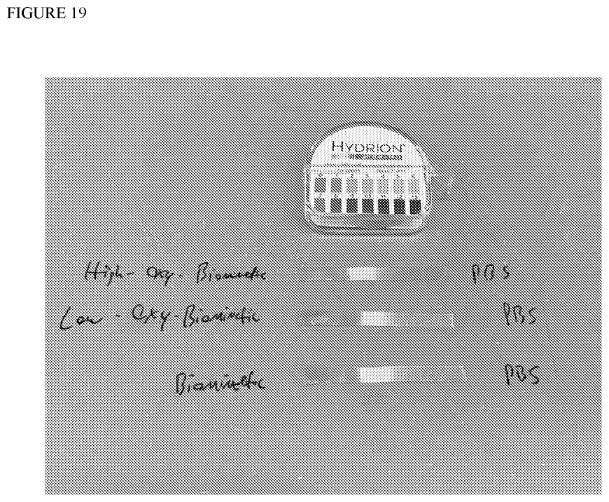

In a sealed beaker, ECM particles (e.g., porcine small intestinal submucosa, porcine urinary bladder matrix, etc.; and combinations thereof) are digested with pepsin from porcine gastric mucosa in dilute hydrochloric acid at room temperature for 24 h while stirring, forming a digested ECM solution. The solution is neutralized with dilute sodium hydroxide and calcium phosphates are added, as described in previous embodiments. Then, an amount of oxysterol or combination of oxysterols is added to the solution. It is anticipated that the ratio of concentration of oxysterol to ECM can be about 10:1. In other words, the range of oxysterol amounts can vary from about 1% of the weight of ECM to about 1000% of the weight of ECM. After about three minutes of stirring, a homogenous mixture comprising ECM, calcium phosphates, and oxysterols—and potentially any additional mineral and/or pharmaceutical agent—has been achieved, and the solution has begun to thicken. The mixture is poured into a mold, which is then placed into an incubator at 37° C. The thickened material is lyophilized, thereby forming a solid, porous, homogenously dispersed mixture comprising ECM, calcium phosphates, and oxysterols—and potentially any additional mineral and/or pharmaceutical agent—from a one-pot liquid ECM solution at room temperature and neutral pH (FIGS. 18-19).

The material is then submerged in a solution of dopamine in tris buffer at pH 8.5, which infuses the material with polydopamine. This material is then lyophilized to create a bone graft of the present invention, namely, a solid, porous, homogenously dispersed mixture of ECM, calcium phosphates, and oxysterols—and potentially any additional mineral and/or pharmaceutical agent—that has been infused with polydopamine. This material may be subsequently submerged in a tris buffer solution at pH 8.5 containing growth factors (e.g., BMP-2, VEGF-165, PDGF-BB), loading growth factors onto the material. Lyophilizing this material creates a growth factor-loaded version of a bone graft of the present invention. In some embodiments, the dosage of BMP-2 is between about 1 μg to about 1000 μg, including, for example, dosages of 1, 2, 3, 4, 5, 6, 7, 8, 9 10 μg or more, such as 200, 300 400, 500, 600, 700, 800, 900, to 1000 μg.

Comparison of Bone Graft Materials

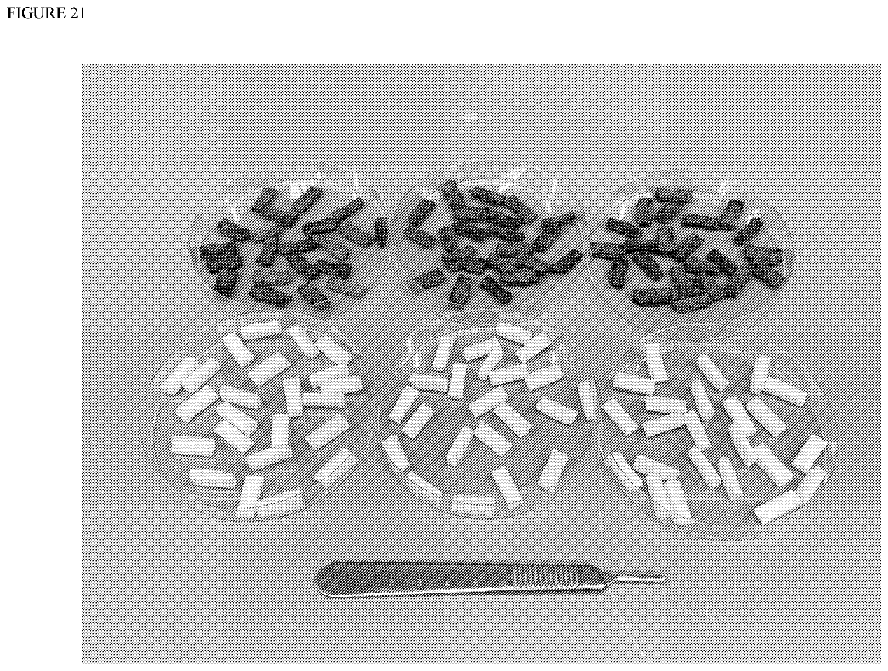

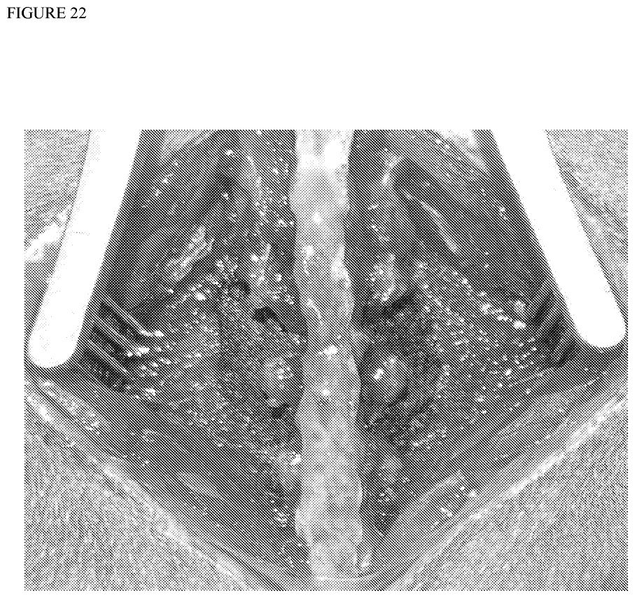

A bone graft material of the present invention was made according to the methods as described above, comprising a solid, porous, homogenously dispersed mixture of ECM and calcium phosphates that was infused with polydopamine. The bone graft material of the present invention was compared directly against the collagen sponge in the Infuse™ Bone Graft in a rat model of posterolateral inter-transverse process spinal fusion of the lumbar vertebrae as described above. The purpose was to compare the spinal fusion ability of the bone graft material of the present invention to that of the Infuse™ Bone Graft collagen sponge. FIG. 21 shows the bone graft material of the present invention (dark pieces) compared to the Infuse™ Bone Graft collagen sponge (white pieces). Different amounts of BMP-2, which was obtained from the Infuse™ Bone Graft, were added in solution form to either the bone graft of the present invention or the collagen sponge 15 minutes before implantation, which is analogous to the clinical use of the Infuse™ Bone Graft. FIG. 22 shows the implantation of the bone graft material of the present invention in this way. Sixty rats were used in total, split equally between the graft types. Specifically, the following protocol was used, involving six different groups:
1. Bone graft material of present invention+20 μg BMP-2
2. Bone graft material of present invention+2 μg BMP-2
3. Bone graft material of present invention+0.2 μg BMP-2
4. Collagen sponge+20 μg BMP-2 (positive control)
5. Collagen sponge+2 μg BMP-2
6. Collagen sponge+0.2 μg BMP-2 (negative control)

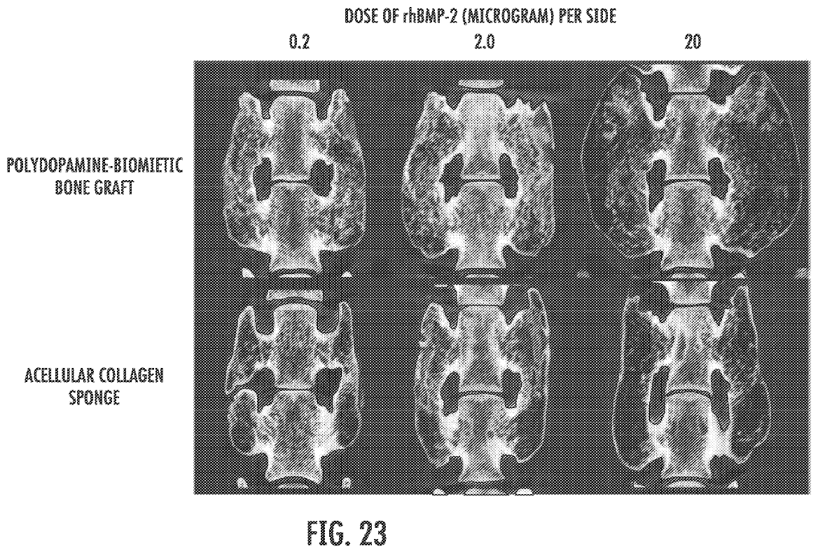

As shown in FIG. 23, both the bone graft material of the present invention and the collagen sponge lead to 100% fusion rates when using 20 μg of or 2 μg of rhBMP-2.

However, at the ultra-low dose of 0.2 μg rhBMP-2, the collagen sponge group shows a non-union (“cleft,” on the left side), whereas the polydopamine-infused biomimetic bone graft of the present invention produces fusion.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A biomimetic bone graft material comprising one or more calcium phosphate particles, extracellular matrix (ECM) particles comprising one or more of biomolecules including collagens, elastins, glycosaminoglycans, proteoglycans, glycoproteins, matricellular proteins, and a combination thereof in a mixture.

2. The biomimetic bone graft material of claim 1, wherein the one or more calcium phosphate particles have a diameter of 200 μm or less.

3. The biomimetic bone graft material of claim 1, wherein the calcium phosphate particles are selected from the group consisting of monocalcium phosphate, dicalcium phosphate, tricalcium phosphate, tetracalcium phosphate, octacalcium phosphate, amorphous calcium phosphate, dicalcium diphosphate, calcium triphosphate, apatite, calcium pyrophosphate, silicate-substituted calcium phosphate, bioactive glass, and a combination thereof.

4. The biomimetic bone graft material of claim 1, wherein the ECM particles have a diameter of 700 μm or less.

5. The biomimetic bone graft material of claim 1, further comprising a growth factor.

6. The biomimetic bone graft material of claim 5, wherein the growth factor is selected from the group consisting of BMPs, calcitonin, parathyroid hormone, AB204, angiopoietin, erythropoietin, basic fibroblast growth factor, transforming growth factor betaulin, NEL-like protein 1, peptide B2A, insulin like growth factor, vascular endothelial growth factor, platelet derived growth factor, and a combination thereof.

7. The biomimetic bone graft material of claim 1, further comprising polydopamine.

8. A method of making a biomimetic bone graft material comprising the steps of:
a) digesting extracellular matrix (ECM) particles comprising one or more of glycosaminoglycans, proteoglycans, glycoproteins, matricellular proteins, and a combination thereof with a protease resulting in an ECM solution;
b) neutralizing the ECM solution of a);
c) adding calcium phosphate particles to the mixture forming a homogeneously dispersed mixture; and
d) incubating the mixture until it solidifies.

9. The method of claim 8, further comprising the step of:
e) contacting the material with a solution of dopamine forming a polydopamine-activated bone graft material comprising an extracellular matrix.

10. The method of claim 9, further comprising the step of:
f) lyophilizing the polydopamine-activated bone graft material comprising an extracellular matrix.

11. The method of claim 8, further comprising the step of contacting the bone graft material comprising an extracellular matrix and calcium phosphate with a solution containing a growth factor forming a bone graft material with a growth factor.

12. The method of claim 8, further comprising the step of lyophilizing the bone graft material with a growth factor.

13. The method of claim 8, wherein the growth factor is selected from the group consisting of BMPs, calcitonin, parathyroid hormone, AB204, angiopoietin, erythropoietin, basic fibroblast growth factor, transforming growth factor beta, insulin, NEL-like protein 1, peptide B2A, insulin-like growth factor, vascular endothelial growth factor, platelet derived growth factor, and combinations thereof.

14. The method of claim 13, wherein the growth factor is selected from the group consisting of BMP-2, VEGF-165, PDGF-BB, and a combination thereof.

15. The method of claim 8, further comprising the step of adding a pharmaceutical agent or mineral before pouring the homogeneously dispersed mixture into a mold.

16. The method of claim 8 wherein the protease is selected from the group consisting of pepsin, chymotrypsin, matrix metalloproteinase, collagenase, alcalase, papain, cathepsin, trypsin, or a combination thereof.

17. The method of claim 7 wherein the pepsin is derived from porcine gastric mucosa.

18. The method of claim 8, further comprising the step of pouring the homogeneously dispersed mixture into a mold.

19. The method of claim 8, wherein the ECM solution is neutralized by adding a dilute base of sodium hydroxide.

20. The method of claim 8, wherein the incubation is at 37° C. degrees.

21. The method of claim 8, wherein the bone graft material is substantially free of water or dry.

22. The method of claim 8, wherein calcium phosphate particles have a size less than 200 μm.

23. The method of claim 8, wherein the material the weight ratio of calcium phosphate to ECM is about 2:1.

24. The method of claim 8, wherein the digestion of ECM particles occurs using 10 mg/ml of the ECM particle, 1 mg/mL of pepsin, and 0.01N HCl for 24 hours.

25. A method for treating a subject having a bone injury or condition requiring a bone graft comprising administering to the subject the biomimetic bone graft material of claim 1.

26. The method of claim 25, wherein the injury is selected from the group consisting of a fracture; a knee problem requiring a knee replacement; a hip problem requiring a hip replacement; a missing tooth requiring bone for a dental implant; a bone issue requiring the treatment of critical-sized bony defects; a bone problem requiring a fusion procedure comprising a foot fusion, an ankle fusion, a finger fusion, a wrist fusion, a spinal fusion; and a combination thereof.
